(19) United States
(12) Patent Application Publication
(10) Pub. No.: US 2012/0142022 A1
(43) Pub. Date: Jun. 7, 2012

(54) GLYCOSYLATED MAMMALIAN NGAL AND USE THEREOF

(75) Inventors: Bailin Tu, Libertyville, IL (US); Joan D. Tyner, Beach Park, IL (US); Lowell J. Tyner, Chicago, IL (US)

(73) Assignee: ABBOTT LABORATORIES, Abbott Park, IL (US)

(21) Appl. No.: 13/354,312

(22) Filed: Jan. 19, 2012

Related U.S. Application Data
(63) Continuation of application No. 12/329,301, filed on Dec. 5, 2008, now abandoned.
Application No. 12/329,301 is a continuation of application No. PCT/US08/80325, filed on Oct. 17, 2008.
Application No. PCT/US08/80325 is a continuation-in-part of application No. 12/104,408, filed on Apr. 16, 2008, now abandoned.
Application No. 12/104,408 is a continuation-in-part of application No. 12/104,413, filed on Apr. 16, 2008.
Application No. 12/104,413 is a continuation-in-part of application No. 12/104,410, filed on Apr. 16, 2008.
(60) Provisional application No. 60/981,470, filed on Oct. 19, 2007.
(60) Provisional application No. 60/981,473, filed on Oct. 19, 2007.
(60) Provisional application No. 60/981,471, filed on Oct. 19, 2007.

(51) Int. Cl. G01N 33/53; C07H 21/04; G01N 33/566; C07K 14/435; C12P 21/06; C12N 5/07

(52) U.S. Cl. 435/7.1; 435/69.1; 435/352; 435/358; 436/501; 530/350; 530/402; 536/23.5

(57) ABSTRACT

The present invention relates to glycosylated mammalian NGAL, and methods of using said glycosylated mammalian NGAL.

### CROSS REFERENCE TO RELATED APPLICATIONS

This application is a Continuation of copending application Ser. No. 12/329,301, filed Dec. 5, 2008, which is a continuation of international application no. PCT/US08/80325 filed Oct. 17, 2008, which is a continuation-in-part application of U.S. nonprovisional application Ser. Nos. 12/104,408, 12/104,410, and 12/104,413, all filed on Apr. 16, 2008, which claim priority to U.S. provisional application Nos. 60/981,470, 60/981,471 and 60/981,473, all filed on Oct. 19, 2007 (now all expired), all of which are incorporated herein by reference in their entireties.

### TECHNICAL FIELD

The present invention relates to glycosylated mammalian NGAL, and to methods of using the glycosylated mammalian NGAL.

### BACKGROUND

Lipocalins are a family of extracellular ligand-binding proteins that are found in a variety of organisms from bacteria to humans. Lipocalins possess many different functions, such as the binding and transport of small hydrophobic molecules, nutrient transport, cell growth regulation, and modulation of the immune response, inflammation and prostaglandin synthesis. Moreover, some lipocalins are also involved in cell regulatory processes and serve as diagnostic and prognostic markers in a variety of disease states. For example, the plasma level of alpha glycoprotein is monitored during pregnancy and in diagnosis and prognosis of conditions including cancer chemotherapy, renal dysfunction, myocardial infarction, arthritis, and multiple sclerosis.

The novel lipocalin neutrophil gelatinase-associated lipocalin (or NGAL, also known as Lipocalin-2 or LCN2) from human neutrophils was identified in 1993. NGAL is a 25-kDa lipocalin that exists in monomeric and homo- and heterodimeric forms, the latter as a 46-kDa dimer with human neutrophil gelatinase. A trimer form of NGAL has also been identified. NGAL is secreted from specific granules of activated human neutrophils. Homologous proteins have been identified in mouse (24p3/uterocalin) and rat (alpha (2)-microglobulin-related protein/neu-related lipocalin). Structural data have confirmed a typical lipocalin fold of NGAL with an eight-stranded beta-barrel, but with an unusually large cavity lined with more polar and positively charged amino acid residues than normally seen in lipocalins. The 25-kDa NGAL protein is believed to bind small lipophilic substances such as bacteria-derived lipopolysaccharides and formylpeptides, and may function as a modulator of inflammation.

Renal injuries or disease, such as acute kidney failure or chronic kidney failure, can result from a variety of different causes (such as illness, injury, and the like). The early identification and treatment of renal injuries and disease would be useful in preventing disease progression. Currently, serum creatinine is frequently used as a biomarker of kidney function. However, serum creatinine measurements are influenced by muscle mass, gender, race and medications. Unfortunately, these limitations often result in the diagnosis of kidney disease only after significant damage has already occurred.

NGAL is an early marker for acute renal injury or disease. In addition to being produced by specific granules of activated human neutrophils, NGAL is also produced by nephrons in response to tubular epithelial damage and is a marker of tubulointerstitial (TI) injury. NGAL levels rise in acute tubular necrosis (ATN) from ischemia or nephrotoxicity, even after mild “subclinical” renal ischemia, as compared to normal serum creatinine levels. Moreover, NGAL is known to be expressed by the kidney in cases of chronic kidney disease (CKD). Elevated urinary NGAL levels have been suggested as predictive of progressive kidney failure. It has been previously demonstrated that NGAL is markedly expressed by kidney tubules very early after ischemic or nephrotoxic injury in both animal and human models. NGAL is rapidly secreted into the urine, where it can be easily detected and measured, and precedes the appearance of any other known urinary or serum markers of ischemic injury. The protein is resistant to proteases, suggesting that it can be recovered in the urine as a faithful marker of tubule expression of NGAL. Further, NGAL derived from outside of the kidney, for example, filtered from the blood, does not appear in the urine, but rather is quantitatively taken up by the proximal tubule.

A variety of immunoassays are known in the art for detecting NGAL. As mentioned previously herein, NGAL is found as a monomer, as a dimer (a homodimer or heterodimer) and even as a trimer. Thus, there is a need in the art for new antibodies and immunoassays which are able to specifically detect and distinguish between NGAL monomer, dimer or trimer in a test sample. Additionally, there is also a need in the art for immunoassays that are able to quantify the relative proportion of monomer to dimer contained in a test sample. Such new antibodies and immunoassays can be used to assess among other things the extent of any renal injury or disease in a patient, monitor the kidney status of a patient suffering from renal injury or disease, or assess the extent of any renal injury in a patient and thereafter monitor the patient's kidney status. Of course, necessary for such immunoassay are the appropriate polypeptides that can be employed, e.g., either as calibrators/controls, and/or as immunogens for making antibodies of interest. Additional objects and advantages of the invention will be apparent from the description provided herein.

### SUMMARY

In one embodiment, the present invention relates to a Chinese Hamster Ovary (CHO) cell line which produces glycosylated mammalian NGAL. Specifically, the mammalian glycosylated NGAL is selected from the group consisting of: canine, feline, rat, murine, horse, non-human primates and humans. If the glycosylated mammalian NGAL is human NGAL, then the glycosylated mammalian NGAL is wild-type human NGAL. Moreover, the glycosylated wild-type human NGAL comprises a molecular weight of about 25 kDa. If the glycosylated mammalian NGAL is wild-type NGAL, then the wild-type NGAL comprises the amino acid sequence of SEQ ID NOS:1 or 12. An example of a CHO cell line which produces glycosylated mammalian NGAL is the CHO cell line having ATCC Accession No. PTA-8020.

In the CHO cell line described herein, the glycosylated mammalian NGAL comprises an amino acid sequence that comprises one or more amino acid substitutions, deletions, or additions when compared to the amino acid sequence of wild-type mammalian NGAL. Specifically, the glycosylated mammalian NGAL is human NGAL, and further, the human NGAL comprises an amino acid substitution at the amino acid corresponding to amino acid 87 of the amino acid sequence of wild-type human NGAL set forth in SEQ ID NOS:1 or 12. Specifically, the amino acid substitution substitution comprises replacement of a cysteine with a serine. More specifically, the glycosylated human NGAL comprises the amino acid sequence of SEQ ID NOS:2 or 10. An example of a CHO cell line comprises an amino acid substitution at the amino acid corresponding to amino acid 87 of the amino acid sequence of wild-type human NGAL is the CHO cell line having ATCC Accession No. PTA-8168.

In another embodiment, the present invention relates to a human embryonic kidney (HEK) cell line which produces glycosylated mammalian NGAL. Specifically, the mammalian glycosylated NGAL is selected from the group consisting of: canine, feline, rat, murine, horse, non-human primates and humans. The glycosylated mammalian NGAL is wild-type human NGAL. If the glycosylated mammalian NGAL is wild-type NGAL, then the wild-type NGAL comprises the amino acid sequence of SEQ ID NOS:1 or 12.

In the HEK cell line described herein, the glycosylated mammalian NGAL can comprise an amino acid sequence that comprises one or more amino acid substitutions, deletions, or additions when compared to the amino acid sequence of wild-type mammalian NGAL. Specifically, the glycosylated mammalian NGAL is human NGAL, and further, the human NGAL comprises an amino acid substitution at the amino acid corresponding to amino acid 87 of the amino acid sequence of wild-type human NGAL set forth in SEQ ID NOS:1 or 12. Specifically, the amino acid substitution substitution comprises replacement of a cysteine with a serine.

In another embodiment, the present invention relates to a method of producing glycosylated mammalian NGAL. The method can comprise the steps of:

(a) transfecting a cell line with a gene encoding mammalian NGAL under conditions such that glycosylated mammalian NGAL is produced; and

(b) recovering the glycosylated mammalian NGAL produced by the cell line.

In the above method, the mammalian glycosylated NGAL is selected from the group consisting of: canine, feline, rat, murine, horse, non-human primates and humans. Specifically, the glycosylated mammalian NGAL is human NGAL.

In the above method, the cell line comprises Chinese Hamster Ovary (CHO) cells. Alternatively, the cell line comprises human embryonic kidney cells.

The above method also further comprises in step (a), transfecting the cell line with an amplification gene, carrying out selection for amplified cells, and then carrying out step (b). The amplification gene encodes dihydrofolate reductase or glutamine synthase, and selection is done with methotrexate or glutamine.

In the above method, the glycosylated human glycosylated human NGAL comprises wild-type human NGAL. Specifically, the wild-type human NGAL comprises the amino acid sequence of SEQ ID NOS:1 or 12. Moreover, the glycosylated wild-type human NGAL can comprise a molecular weight of about 25 kDa.

In the above method, the glycosylated mammalian NGAL can comprise an amino acid sequence that comprises one or more amino acid substitutions, deletions, or additions when compared to the amino acid sequence of wild-type mammalian NGAL. Specifically, the glycosylated mammalian NGAL is human NGAL, and further, the human NGAL comprises an amino acid substitution at the amino acid corresponding to amino acid 87 of the amino acid sequence of wild-type human NGAL set forth in SEQ ID NOS:1 or 12. Specifically, the amino acid substitution substitution comprises replacement of a cysteine with a serine. More specifically, the glycosylated human NGAL comprises the amino acid sequence of SEQ ID NOS:2 or 10.

In another embodiment, the present invention relates to glycosylated human NGAL produced by the above method, wherein the human NGAL comprises the sequence of SEQ ID NOS:1 or 12.

In yet another embodiment, the present invention relates to glycosylated human NGAL produced by the above method, wherein the human NGAL comprises the sequence of SEQ ID NOS:2 or 10.

In still yet another embodiment, the present invention relates to an isolated mutant glycosylated human NGAL comprising the sequence of SEQ ID NOS:2 or 10.

In still yet another embodiment, the present invention relates to a calibrator or control for use in an assay for detecting mammalian NGAL in a test sample, the calibrator or control comprising glycosylated mammalian NGAL. The mammalian NGAL to be detected is canine, feline, murine, horse, non-human primates and humans. Moreover, the mammalian NGAL is glycosylated human NGAL comprising the sequence of SEQ ID NOS:2 or 10. Alternatively, the mammalian NGAL is glycosylated human NGAL comprising the sequence of SEQ ID NOS:1 or 12.

In still yet another embodiment, the present invention relates to a method of preventing or eliminating the formation of at least one dimer of human NGAL in a calibrator, control or other sample. The method comprises introducing an amino acid substitution into the human NGAL which comprises replacement of cysteine with serine at the amino acid corresponding to amino acid 87 of the amino acid sequence of wild-type human NGAL set forth in SEQ ID NOS:1 or 12. In the above method, the dimer is a homodimer. Alternatively, the dimer is a heterodimer.

In still yet another embodiment, the present invention relates to an isolated and purified human NGAL polynucleotide comprising the sequence of SEQ ID NOS:4 or 11.

### BRIEF DESCRIPTION OF THE FIGURES

FIG. 1 shows the human NGAL wild-type antigen sequence (SEQ ID NO:1). Native human NGAL signal peptide residues are in italics and underlined. Wild-type human NGAL sequences in pJV-NGAL-A3 plasmid are in bold. The 6×His tag in the C-terminal is underlined.

FIG. 2 shows plasmid pJV-NGAL-A3 (also known as pJV-NGAL-hisA) containing the wild-type human NGAL sequence as described in Example 1.

FIG. 3 shows human NGAL wild-type antigen production yield (ordinate) using various transfected Chinese Hamster Ovary (CHO) clones (abscissa) as described in Example 1. The clones were: (A) CHO clone #204, which exhibited a yield of 3.8 mg/L; (B) CHO clone #465, which was amplified by 20 nM methotrexate (MTX) and exhibited a yield of about 4 mg/L; (C) CHO clone #950, which was amplified by 100 nM MTX and exhibited a yield of about 32 mg/L; (D) CHO clone #113, which was amplified by 500 nM of MTX and exhibited a yield of about 88 mg/L; and (E) CHO clone #662, which was amplified by 5 μM MTX and exhibited a yield of about 129 mg/L.

FIG. 4 shows a SDS-PAGE gel performed according to Example 2 and which shows that human NGAL wild-type antigen from CHO clone #662 was converted from a dimer to a monomer under reducing conditions (Lane 4). Lanes: (1) marker; (2) recombinant NGAL from CHO Clone #662, non-reducing and no boiling conditions; (3) recombinant NGAL from CHO Clone #662, non-reducing and boiling conditions; and (4) recombinant NGAL from CHO Clone #662, reducing and boiling conditions.

FIG. 5 shows results of an iron binding assay used to characterize human NGAL wild-type activity by its ability to bind iron (III) dihydroxybenzoic acid (Fe(DHBA)₃) (abscissa) as measured by fluorescence (ordinate). The results show that human NGAL expressed from HEK293 produced according to Example 2 can bind Fe(DHBA)₃ greater than 1.5 μM.

FIG. 6 shows a SDS-PAGE gel which confirms that recombinant human NGAL antigen purified from HEK cells as described herein can bind to commercially available anti-human monoclonal antibodies: (A) HYB 211-01; (B) HYB 211-02; and (C) HYB 211-05. Lanes: (1) reduced recombinant NGAL (See Examples, 2 μg); (2) reduced recombinant NGAL (R&D Systems, 2 μg); (3) reduced human HNL (Diagnostics Development, Uppsala, Sweden; native NGAL, 2 μg); (4) non-reduced recombinant NGAL (See Examples, 2 μg); (5) non-reduced recombinant NGAL (R&D Systems, Minneapolis, Minn., 2 μg); (6) non-reduced human HNL (Diagnostics Development, Uppsala, Sweden; native NGAL, 2 μg); (7) protein marker.

FIG. 7 is a MALDI MS spectrum taken after 72 hours of PNGase treatment and which confirms that the CHO cells express human wild-type NGAL that demonstrates N-linked glycan.

FIG. 8 shows plasmid pJ-NGAL(C87S)-his A (also known as pJV-NGAL(ser87)-His-T3) containing a human NGAL with a C87S mutation.

FIG. 9 shows the human NGAL C87S mutant antigen sequences (SEQ ID NO:2). Native human NGAL signal peptides are in italics and underlined. Wild-type NGAL sequences in the pJV-NGAL(Ser87)-His-T3 plasmid are in bold, and the NGAL C87S mutant codon sequence is in bold and underlined. The 6×His tag in the C-terminal is also underlined.

FIG. 10 shows a SDS-PAGE gel which confirms that for CHO cells expressing C87S mutant NGAL antigen, greater than about 95% of the NGAL is in monomer form (with or without reducing agent added) and no dimer human NGAL was detected. Lanes: (1) marker (Invitrogen Corp., Carlsbad, Calif.); (2) NGAL C87S mutant, non-reducing conditions; (3) NGAL C87S mutant, reducing conditions; (4) wild-type NGAL, non-reducing conditions; and (5) wild-type NGAL, reducing conditions.

FIG. 11 shows the wild-type human NGAL polynucleotide sequence (SEQ ID NO:3).

FIG. 12 shows the mutant human NGAL polynucleotide sequence (SEQ ID NO:4).

### DETAILED DESCRIPTION

Certain glycosylated mammalian NGAL proteins have been discovered. These NGAL proteins alone or in or in combination with antibodies directed against the NGAL proteins have a variety of uses, for example, as a component of a diagnostic assay, or present in an immunoassay kit, or as immunogens for making antibodies in improved immunoassays.

### A. Definitions

As used herein, the singular forms “a,” “an” and “the” include plural referents unless the context clearly dictates otherwise. For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

### a) Antibody

As used herein, the terms “antibody” and “antibodies” refer to monoclonal antibodies, multispecific antibodies, human antibodies, humanized antibodies (fully or partially humanized), animal antibodies (in one aspect, a bird (for example, a duck or goose), in another aspect, a shark or whale, in yet another aspect, a mammal, including a non-primate (for example, a cow, pig, camel, llama, horse, goat, rabbit, sheep, hamsters, guinea pig, feline, canine, rat, mouse, etc) and a non-human primate (for example, a monkey, such as a cynomologous monkey, a chimpanzee, etc), recombinant antibodies, chimeric antibodies, single-chain Fvs (scFv), single chain antibodies, single domain antibodies, Fab fragments, F(ab′)₂ fragments, disulfide-linked Fv (sdFv), and anti-idiotypic (anti-Id) antibodies (including, for example, anti-Id antibodies to antibodies of the present invention), and functionally active epitope-binding fragments of any of the above. In particular, antibodies include immunoglobulin molecules and immunologically active fragments of immunoglobulin molecules, namely, molecules that contain an antigen binding site. Immunoglobulin molecules can be of any type (for example, IgG, IgE, IgM, IgD, IgA and IgY), class (for example, IgG₁, IgG₂, IgG₃, IgG₄, IgA₁ and IgA₂) or subclass. For simplicity sake, an antibody against an analyte is frequently referred to as being either an “anti-analyte antibody”, or merely an “analyte antibody” (e.g., an NGAL antibody).

Antibodies directed against the polypeptides as described herein, and methods of making such antibodies using the polypeptides are described in U.S. Provisional Application Ser. No. 60/981,471 filed Oct. 19, 2007 (incorporated by reference for its teachings regarding same). Furthermore, the use of such antibodies as well as the polypeptides of the present invention, e.g., in immunoassays and/or as calibrators, controls, and immunodiagnostic agents, are described in U.S. Provisional Application Ser. No. 60/981,473 filed Oct. 19, 2007 (incorporated by reference for its teachings regarding same).

### b) Renal Tubular Cell Injury

As used herein the expression “renal tubular cell injury” means a renal or kidney failure or dysfunction, either sudden (acute) or slowly declining over time (chronic), that can be triggered by a number of disease or disorder processes. Both acute and chronic forms of renal tubular cell injury can result in a life-threatening metabolic derangement.

### c) Acute Kidney Disease

An “acute renal tubular cell injury” means acute ischemic renal injury (IRI) or acute nephrotoxic renal injury (NRI). IRI includes but is not limited to ischemic injury and chronic ischemic injury, acute renal failure, acute glomerulonephritis, and acute tubulo-interstitial nephropathy. NRI toxicity includes but is not limited to, sepsis (infection), shock, trauma, kidney stones, kidney infection, drug toxicity, poisons or toxins, or after injection with a radiocontrast dye.

### d) Chronic Kidney Disease

The phrases “chronic renal tubular cell injury”, “progressive renal disease”, “chronic renal disease (CRD)”, “chronic kidney disease (CKD)” as used interchangeably herein, include any kidney condition or dysfunction that occurs over a period of time, as opposed to a sudden event, to cause a gradual decrease of renal tubular cell function or worsening of renal tubular cell injury. One endpoint on the continuum of chronic renal disease is “chronic renal failure (CRF)”. For example, chronic kidney disease or chronic renal injury as used interchangeably herein, includes, but is not limited to, conditions or dysfunctions caused by chronic infections, chronic inflammation, glomerulonephritides, vascular diseases, interstitial nephritis, drugs, toxins, trauma, renal stones, long standing hypertension, diabetes, congestive heart failure, nephropathy from sickle cell anemia and other blood dyscrasias, nephropathy related to hepatitis, HIV, parvovirus and BK virus (a human polyomavirus), cystic kidney diseases, congenital malformations, obstruction, malignancy, kidney disease of indeterminate causes, lupus nephritis, membranous glomerulonephritis, membranoproliferative glomerulonephritis, focal glomerular sclerosis, minimal change disease, cryoglobulinemia, Anti-Neutrophil Cytoplasmic Antibody (ANCA)-positive vasculitis, ANCA-negative vasculitis, amyloidosis, multiple myeloma, light chain deposition disease, complications of kidney transplant, chronic rejection of a kidney transplant, chronic allograft nephropathy, and the chronic effects of immunosuppressives. Preferably, chronic renal disease or chronic renal injury refers to chronic renal failure or chronic glomerulonephritis.

### e) Immunodiagnostic Reagent

An “immunodiagnostic reagent” comprises one or more antibodies that specifically bind to a region of an NGAL protein as described herein. Immunodiagnostic agents, are described in U.S. Provisional Application Ser. No. 60/981,473 filed Oct. 19, 2007 (incorporated by reference for its teachings regarding same).

### f) NGAL Polynucleotide and Polypeptide Sequences

The NGAL can be any NGAL sequence, e.g., including that set forth as Genbank accession numbers Genpept CAA58127 (SEQ ID NO:1), AAB26529, XP_{—}862322, XP_{—}548441, P80108, P11672, X83006.1, X99133.1, CAA67574.1, BC033089.1, AAH33089.1, S75256.1, AD14168.1, JC2339, 1DFVA, 1DFVB, 1L6MA, 1L6 MB, 1L6MC, 1NGLA, 1QQSA, 1X71A, 1X71B, 1X71C, 1X89A, 1X89B, 1X89C, 1X8UA, 1X8UB, and 1X8UC. NGAL polynucleotide and polypeptide (e.g., polyamino acid) sequences are as found in nature, based on sequences found in nature, isolated, synthetic, semi-synthetic, recombinant, or other. In one embodiment, the NGAL is human NGAL (also known as “hNGAL”). Unless specified otherwise, NGAL polypeptide sequences are numbered according to the mature human NGAL sequence minus the 20 residue amino acid signal peptide typically found in nature (and minus any other signal peptide sequence). When a signal peptide is present, it is numbered with negative numbers, e.g., as residues −1 to −20, with comparable numbering applied for the encoding polynucleotide sequence.

Likewise, an initial Met residue at the N-terminus of NGAL is present only in NGAL produced in prokaryotes (e.g., E. coli), or in synthetic (including semi-synthetic) or derived sequences, and not in NGAL produced in eukaryotes (e.g., mammalian cells, including human and yeast cells). Consequently, when present, an initial Met residue is counted herein as a negative number, e.g., as residue −1, with no similar numbering adjustment being made for the polynucleotide sequence in a prokaryotic versus eukaryotic background or expression system inasmuch as the polynucleotide sequence is replicated and transcribed the same in both backgrounds and the difference lies at the level of translation.

Accordingly, the disclosure herein encompasses a multitude of different NGAL polynucleotide and polypeptide sequences as present and/or produced in a prokaryotic and/or eukaryotic background (e.g., with consequent optimization for codon recognition). In sum, the sequences may or may not possess or encode: (a) a signal peptide; (b) an initiator Met residue present in the mature NGAL sequence at the N-terminus; (c) an initiator Met residue present at the start of a signal peptide that precedes the mature NGAL protein; and (d) other variations such as would be apparent to one skilled in the art.

Exemplary sequences include, but are not limited to, those as set forth herein: SEQ ID NO: (NGAL wild-type polypeptide including signal peptide); SEQ ID NO:2 (NGAL mutant polypeptide including signal peptide); SEQ ID NO:10 (NGAL mutant polypeptide not including any signal peptide, and which can be preceded by a Met initiator residue when produced in prokaryotes and a Met initiator codon is present; however, there is no Met initiator residue when produced in eukaryotes, regardless of whether a Met initiator codon is present); SEQ ID NO:12 (NGAL wild-type polypeptide not including any signal peptide, and which can be preceded by a Met initiator residue when produced in prokaryotes and a Met initiator codon is present; however, there is no Met initiator residue when produced in eukaryotes, regardless of whether a Met initiator codon is present); SEQ ID NO:3 (NGAL wild-type polynucleotide sequence including that encoding a signal peptide); SEQ ID NO:4 (NGAL mutant polynucleotide including that encoding a signal peptide); SEQ ID NO:11 (NGAL mutant polynucleotide, synthetic or for eukaryotic expression, not including that encoding any signal peptide, but which optionally further can be preceded at the N-terminus either with or without a Met initiator codon, e.g., ATG); SEQ ID NO:9 (NGAL mutant polynucleotide, synthetic or for prokaryotic expression, not including that encoding any signal peptide, but which optionally further can be preceded at the N-terminus either with or without a Met initiator codon, e.g., ATG).

### g) Glycosylated Mammalian NGAL

As used herein, the phrases “oligosaccharide moiety” or “oligosaccharide molecule” as used interchangeably herein refers to a carbohydrate-containing molecule comprising one or more monosaccharide residues, capable of being attached to a polypeptide (to produce a glycosylated polypeptide, such as, for example, mammalian NGAL) by way of in vivo or in vitro glycosylation. Except where the number of oligosaccharide moieties attached to the polypeptide is expressly indicated, every reference to “oligosaccharide moiety” referred to herein is intended as a reference to one or more such moieties attached to a polypeptide. Preferably, the polypeptide to which said carbohydrate-containing molecule is capable of being attached is wild-type or mutant mammalian NGAL, i.e., to provide “glycosylated mammalian NGAL” as described further herein.

The term “in vivo glycosylation” is intended to mean any attachment of an oligosaccharide moiety occurring in vivo, for example, during posttranslational processing in a glycosylating cell used for expression of the polypeptide, for example, by way of N-linked and O-linked glycosylation. Usually, the N-glycosylated oligosaccharide-moiety has a common basic core structure composed of five monosaccharide residues, namely two N-acetylglucosamine residues and three mannose residues. The exact oligosaccharide structure depends, to a large extent, on the glycosylating organism in question and on the specific polypeptide.

The phrase “in vitro glycosylation” refers to a synthetic glycosylation performed in vitro, normally involving covalently linking an oligosaccharide moiety to an attachment group of a polypeptide, optionally using a cross-linking agent. In vitro glycosylation can be achieved by attaching chemically synthesized oligosaccharide structures to a polypeptide (such as, for example, mammalian NGAL) using a variety of different chemistries. For example, the chemistries that can be employed are those used for the attachment of polyethylene glycol (PEG) to proteins, wherein the oligosaccharide is linked to a functional group, optionally, via a short spacer. In vitro glycosylation can be carried out in a suitable buffer at a pH of about 4.0 to about 7.0 in protein concentrations of about 0.5 to about 2.0 mg/mL in a volume of about 0.02 to about 2.0 ml. Other in vitro glycosylation methods are described, for example in WO 87/05330, by Aplin et al., CRC Crit. Rev. Biochem. 259-306 (1981), by Lundblad et al. in Chemical Reagents for Protein Modification, CRC Press Inc., Boca Raton, Fla., Yan et al., Biochemistry, 23:3759-3765 (1982) and Doebber et al., J. Biol. Chem., 257:2193-2199 (1982).

### h) Human NGAL Fragment

As used herein, the term “human NGAL fragment” herein refers to a polypeptide that comprises a part that is less than the entirety of a mature human NGAL or NGAL including a signal peptide. In particular, a human NGAL fragment comprises from about 5 to about 178 or about 179 contiguous amino acids of SEQ ID NOS:1, 2, 10 or 12. In particular, a human NGAL fragment comprises from about 5 to about 170 contiguous amino acids of SEQ ID NOS:1, 2, 10 or 12. In particular, a human NGAL fragment comprises at least about 5 contiguous amino acids of SEQ ID NO:1, 2, 10 or 12, at least about 10 contiguous amino acids residues of SEQ ID NOS:1, 2, 10 or 12; at least about 15 contiguous amino acids residues of amino acids of SEQ ID NOS:1, 2, 10 or 12; at least about 20 contiguous amino acids residues of SEQ ID NOS:1, 2, 10 or 12; at least about 25 contiguous amino acids residues of SEQ ID NOS:1, 2, 10 or 12, at least about 30 contiguous amino acid residues of amino acids of SEQ ID NOS:1, 2, 10 or 12, at least about 35 contiguous amino acid residues of SEQ ID NOS:1, 2, 10 or 12, at least about 40 contiguous amino acid residues of SEQ ID NOS:1, 2, 10 or 12, at least about 45 contiguous amino acid residues of SEQ ID NOS:1, 2, 10 or 12, at least about 50 contiguous amino acid residues of SEQ ID NOS:1, 2, 10 or 12, at least about 55 contiguous amino acid residues of SEQ ID NOS:1, 2, 10 or 12, at least about 60 contiguous amino acid residues of SEQ ID NOS:1, 2, 10 or 12, at least about 65 contiguous amino acid residues of SEQ ID NOS:1, 2, 10 or 12, at least about 70 contiguous amino acid residues of SEQ ID NOS:1, 2, 10 or 12, at least about 75 contiguous amino acid residues of SEQ ID NOS:1, 2, 10 or 12, at least about 80 contiguous amino acid residues of SEQ ID NOS:1, 2, 10 or 12, at least about 85 contiguous amino acid residues of SEQ ID NOS:1, 2, 10 or 12, at least about 90 contiguous amino acid residues of SEQ ID NOS:1, 2, 10 or 12, at least about 95 contiguous amino acid residues of SEQ ID NOS:1, 2, 10 or 12, at least about 100 contiguous amino acid residues of SEQ ID NOS:1, 2, 10 or 12, at least about 105 contiguous amino acid residues of SEQ ID NOS:1, 2, 10 or 12, at least about 110 contiguous amino acid residues of SEQ ID NOS:1, 2, 10 or 12, at least about 115 contiguous amino acid residues of SEQ ID NOS:1, 2, 10 or 12, at least about 120 contiguous amino acid residues of SEQ ID NOS:1, 2, 10 or 12, at least about 125 contiguous amino acid residues of SEQ ID NOS:1, 2, 10 or 12, at least about 130 contiguous amino acid residues of SEQ ID NOS:1, 2, 10 or 12, at least about 135 contiguous amino acid residues of SEQ ID NOS:1, 2, 10 or 12, at least about 140 contiguous amino acid residues of SEQ ID NOS:1, 2, 10 or 12, at least about 145 contiguous amino acid residues of SEQ ID NOS:1, 2, 10 or 12, at least about 150 contiguous amino acid residues of SEQ ID NOS:1, 2, 10 or 12, at least about 160 contiguous amino acid residues of SEQ ID NOS:1, 2, 10 or 12, at least about 165 contiguous amino acid residues of SEQ ID NOS:1, 2, 10 or 12, at least about 170 contiguous amino acid residues of SEQ ID NOS:1, 2, 10 or 12 or at least about 175 contiguous amino acid residues of SEQ ID NOS:1, 2, 10 or 12.

Examples of human NGAL fragments contemplated by the present invention include, but are not limited to:

(a) a human NGAL fragment of at least about 7 contiguous amino acids which includes amino acid residues 112, 113, 114, 115, 116, 117 and 118 of SEQ ID NOS:1, 2, 10 or 12 (with the numbering of SEQ ID NO:1 and 2 beginning at the Gln residue of the mature sequence immediately following the signal peptide and any Met initiator residue, and the signal peptide and any Met initiator residue(s) being numbered in the negative, as previously described herein);

(b) a human NGAL fragment of at least about 8 contiguous amino acids which includes amino acid residues 112, 113, 114, 115, 116, 117, 118 and 119 of SEQ ID NOS:1, 2, 10 or 12 (with the numbering of SEQ ID NO:1 and 2 beginning at the Gln residue of the mature sequence immediately following the signal peptide and any Met initiator residue);

(c) a human NGAL fragment of at least about 36 contiguous amino acid which includes amino acid residues 112, 118 and 147 of SEQ ID NOS:1, 2, 10 or 12 (with the numbering of SEQ ID NO:1 and 2 beginning at the Gln residue of the mature sequence immediately following the signal peptide and any Met initiator residue);

(d) a human NGAL fragment of at least about 95 contiguous amino acids which includes amino acid residues 15 and 109 of SEQ ID NOS:1, 2, 10 or 12 (with the numbering of SEQ ID NO:1 and 2 beginning at the Gln residue of the mature sequence immediately following the signal peptide and any Met initiator residue);

(e) a human NGAL fragment of at least about 144 contiguous amino acids which includes amino acid residues 15, 109 and 158 of SEQ ID NOS:1, 2, 10 or 12 (with the numbering of SEQ ID NO:1 and 2 beginning at the Gln residue of the mature sequence immediately following the signal peptide and any Met initiator residue);

(f) a human NGAL fragment of at least about 145 contiguous amino acids which includes amino acid residues 15, 109, 158 and 159 of SEQ ID NOS:1, 2, 10 or 12 (with the numbering of SEQ ID NO:1 and 2 beginning at the Gln residue of the mature sequence immediately following the signal peptide and any Met initiator residue); or

(g) a human NGAL fragment of at least about 146 contiguous amino acids which includes amino acid residues 15, 109, 158, 159 and 160 of SEQ ID NOS:1, 2, 10 or 12 (with the numbering of SEQ ID NO:1 and 2 beginning at the Gln residue of the mature sequence immediately following the signal peptide and any Met initiator residue).

Optionally, such human NGAL fragments as described herein are encoded either in part or in the entirety by the corresponding sequences of SEQ ID NOS:3, 4 or 11. Along these lines, in one embodiment, the present invention provides an isolated, purified, or isolated and purified human NGAL polynucleotide comprising or consisting of the sequence of SEQ ID NOS:4 or 11.

### i) NGAL Hybrid

As used herein, the term “NGAL hybrid” or ‘NGAL hybridoma” refers to a particular hybridoma clone or subclone (as specified) that produces an anti-NGAL antibody of interest. Generally, there may be some small variation in the affinity of antibodies produced by a hybridoma clone as compared to those from a subclone of the same type, e.g., reflecting purity of the clone. By comparison, it is well established that all hybridoma subclones originating from the same clone and further, that produce the anti-NGAL antibody of interest produce antibodies of identical sequence and/or identical structure. NGAL hybrids are described in U.S. Provisional Application Ser. No. 60/981,471 filed Oct. 19, 2007 (incorporated by reference for its teachings regarding same).

### j) Epitope

As used herein, the term “epitope”, “epitopes” or “epitopes of interest” refer to a site(s) on any molecule that is recognized and is capable of binding to a complementary site(s) on its specific binding partner. The molecule and specific binding partner are part of a specific binding pair. For example, an epitope can be a polypeptide, protein, hapten, carbohydrate antigen (such as, but not limited to, glycolipids, glycoproteins or lipopolysaccharides) or polysaccharide and its specific binding partner, can be, but is not limited to, an antibody.

In particular, an epitope refers to a particular region (composed of one or more amino acids) of an antigen, namely a protein to which an antibody binds. More specifically, an antigenic epitope is the area on protein surface that interacts with the complementary area (paratope) on the surface of the antibody binding domains. The epitope thus participates in electrostatic interactions, hydrophobic interactions and hydrogen bonding with the antibody and also contains residues responsible for the correct geometry of the surface, its malleability and structural dynamics. There are also buried “second sphere” residues that carry a strong supporting role for the antigenic epitope.

### k) Subject

As used herein, the terms “subject” and “patient” are used interchangeably irrespective of whether the subject has or is currently undergoing any form of treatment. As used herein, the terms “subject” and “subjects” refer to a mammal including, a non-primate (for example, a cow, pig, camel, llama, horse, goat, rabbit, sheep, hamsters, guinea pig, feline, canine, rat, and murine), a non-human primate (for example, a monkey, such as a cynomolgous monkey, chimpanzee, etc) and a human. Preferably, the subject is a human.

### l) Test Sample

As used herein, the term “test sample” refers to a biological sample derived from serum, plasma, blood (including, but not limited to, whole blood), lymph, urine or other bodily fluids of a subject. The test sample can be prepared using routine techniques known to those skilled in the art. Preferably, the test sample is urine or blood.

m) Pretreatment Reagent (e.g., Lysis, Precipitation and/or Solubilization Reagent)

A pretreatment reagent used in a diagnostic assay as described herein is one that lyses any cells and/or solubilizes any analyte that are present in a test sample. Pretreatment is not necessary for all samples, as described further herein. Among other things, solubilizing the analyte (i.e., NGAL) entails release of the analyte from any endogenous binding proteins present the sample. A pretreatment reagent may be homogenous (not requiring a separation step) or heterogeneous (requiring a separation step). With use of a heterogenous pretreatment reagent there is removal of any precipitated analyte binding proteins from the test sample prior to proceeding to the next step of the assay. The pretreatment reagent optionally can comprise: (a) one or more solvents and salt, (b) one or more solvents, salt and detergent, (c) detergent, (d) detergent and salt, or (e) any reagent or combination of reagents appropriate for cell lysis and/or solubilization of analyte. Also, proteases, either alone or in combination with any other pretreament agents (e.g., solvents, detergents, salts, and the like) can be employed.

The terminology used herein is for the purpose of describing particular embodiments only and is not otherwise intended to be limiting.

### B. Glycosylated Mammalian NGAL

The present invention relates to mammalian NGAL of any type (e.g., isolated, recombinant, mutant, wild-type, synthetic, semi-synthetic, and the like), especially mammalian NGAL that optionally is glycosylated, and particularly human NGAL.

In one embodiment, the present invention relates to isolated glycosylated mammalian NGAL. More specifically, the present invention relates to glycosylated mammalian NGAL that contains at least one oligosaccharide molecule or moiety and up to ten (10) oligosaccharide molecules or moieties. The glycosylated mammalian NGAL of the present invention includes, but is not limited to, glycosylated canine NGAL, glycosylated feline NGAL, glycosylated rat NGAL, glycosylated murine NGAL, glycosylated horse NGAL, glycosylated non-human primate NGAL and glycosylated human NGAL. Preferably, the glycosylated mammalian NGAL is human NGAL. Moreover, the glycosylated mammalian NGAL can be wild-type NGAL (namely, any wild-type mammalian NGAL, such as, but not limited to, wild-type canine NGAL, wild-type feline NGAL, wild-type rat NGAL, wild-type murine NGAL, wild-type horse NGAL, wild-type non-human primate NGAL or wild-type human NGAL). Preferably, the wild-type mammalian NGAL, is wild-type human NGAL having the amino acid sequence shown in SEQ ID NO:1 (including a signal peptide, and with the numbering of SEQ ID NO:1 beginning at the Gln residue of the mature sequence immediately following the signal peptide and any Met initiator residue) or SEQ ID NO:12 (not including a signal peptide). Alternatively, the glycosylated mammalian NGAL can be a glycosylated mutant mammalian NGAL that comprises an amino acid sequence comprising one or more amino acid substitutions, deletions or additions when compared to the corresponding amino acid sequence of the wild-type mammalian NGAL. For example, the glycosylated mammalian NGAL can be human NGAL wherein the amino acid sequence of the wild-type human NGAL (See, e.g., SEQ ID NOS:1 or 12) contains at least one amino acid substitution. Specifically, at least one amino acid substitution can be made at amino acid residue 87 of SEQ ID NOS:1 or 12. Specifically, the cysteine at amino acid 87 shown in SEQ ID NOS:1 or 12 can be replaced with a serine (See, e.g., SEQ ID NOS:2 and 10). Other substitutions for amino acids other than serine or cysteine can be made, e.g., glycine or alanine. Moreover, other amino acid substitutions, deletions or additions other than the single amino acid substitution at amino acid 87 of SEQ ID NOS:1 or 12 can be made by those skilled in the art using routine experimentation.

The mammalian NGAL employed herein (e.g., optionally glycosylated) can be made using recombinant DNA technology, by chemical synthesis or by a combination of chemical synthesis and recombinant DNA technology. Specifically, a polynucleotide sequence encoding mammalian NGAL may be constructed by isolating or synthesizing a polynucleotide sequence encoding the mammalian NGAL of interest. As mentioned above, the mammalian NGAL (e.g., optionally glycosylated) can be a wild-type mammalian NGAL or can be a mutant mammalian NGAL containing one more amino acid substitutions, deletions or additions. Such amino acid substitutions, deletions or additions can be made using routine techniques known in the art, such as by mutagenesis (for example, using site-directed mutagenesis in accordance with well known methods, e.g., as described in Nelson and Long, Analytical Biochemistry 180:147-151 (1989), random mutagenesis, or shuffling).

The polynucleotide sequence encoding the mammalian NGAL of interest may be prepared by chemical synthesis, such as by using an oligonucleotide synthesizer, wherein oligonucleotides are designed based on the amino acid sequence of the desired mammalian NGAL (wild-type or mutant), and by preferably selecting those codons that are favored in the host cell in which the recombinant mammalian NGAL will be produced. For example, several small oligonucleotides coding for portions of the desired mammalian NGAL may be synthesized and assembled by polymerase chain reaction (PCR), ligation or ligation chain reaction (LCR). The individual oligonucleotides typically contain 5′ or 3′ overhangs for complementary assembly.

Once assembled (such as by synthesis, site-directed mutagenesis or another method), the polynucleotide sequence encoding the mammalian NGAL of interest may be inserted into a recombinant vector and operably linked to any control sequences necessary for expression of thereof in the desired transformed host cell.

Although not all vectors and expression control sequences may function equally well to express a polynucleotide sequence of interest and not all hosts function equally well with the same expression system, it is believed that those skilled in the art will be able to easily make a selection among these vectors, expression control sequences, optimized codons, and hosts for use in the present invention without any undue experimentation. For example, in selecting a vector, the host must be considered because the vector must be able to replicate in it or be able to integrate into the chromosome. The vector's copy number, the ability to control that copy number, and the expression of any other proteins encoded by the vector, such as antibiotic markers, should also be considered. In selecting an expression control sequence, a variety of factors can also be considered. These include, but are not limited to, the relative strength of the sequence, its controllability, and its compatibility with the polynucleotide sequence encoding the mammalian NGAL, particularly as regards potential secondary structures. Hosts should be selected by consideration of their compatibility with the chosen vector, their codon usage, their secretion characteristics, their ability to fold the polypeptide correctly, their fermentation or culture requirements, their ability (or lack thereof) to glycosylate the protein, and the ease of purification of the products coded for by the nucleotide sequence, etc.

The recombinant vector may be an autonomously replicating vector, namely, a vector existing as an extrachromosomal entity, the replication of which is independent of chromosomal replication (such as a plasmid). Alternatively, the vector can be one which, when introduced into a host cell, is integrated into the host cell genome and replicated together with the chromosome(s) into which it has been integrated.

The vector is preferably an expression vector, in which the polynucleotide sequence encoding the mammalian NGAL is operably linked to additional segments required for transcription of the polynucleotide sequence. The vector is typically derived from plasmid or viral DNA. A number of suitable expression vectors for expression in the host cells mentioned herein are commercially available or described in the literature. Useful expression vectors for eukaryotic hosts, include, but are not limited to, vectors comprising expression control sequences from SV40, bovine papilloma virus, adenovirus and cytomegalovirus. Specific vectors include, pcDNA3.1 (+)\Hyg (Invitrogen Corp., Carlsbad, Calif.) and pCI-neo (Stratagene, La Jolla, Calif., USA). Examples of expression vectors for use in yeast cells include, but are not limited to, the 2μ plasmid and derivatives thereof, the POTI vector (See, U.S. Pat. No. 4,931,373), the pJSO37 vector (described in Okkels, Ann. New York Acad. Sci., 782:202-207, (1996)) and pPICZ A, B or C (Invitrogen Corp., Carlsbad, Calif.). Examples of expression vectors for use in insect cells include, but are not limited to, pVL941, pBG311 (Cate et al., “Isolation of the Bovine and Human Genes for Mullerian Inhibiting Substance And Expression of the Human Gene In Animal Cells” Cell, 45:685-698 (1986), pBluebac 4.5 and pMelbac (both of which are available from Invitrogen Corp., Carlsbad, Calif.). A preferred vector for use in the invention is pJV (available from Abbott Laboratories, Abbott Bioresearch Center, Worcester, Mass.).

Other vectors that can be used allow the polynucleotide sequence encoding the mammalian NGAL to be amplified in copy number. Such amplifiable vectors are well known in the art. These vectors include, but are not limited to, those vector that can be amplified by DHFR amplification (See, for example, Kaufman, U.S. Pat. No. 4,470,461, Kaufman et al., “Construction Of A Modular Dihydrofolate Reductase cDNA Gene: Analysis Of Signals Utilized For Efficient Expression” Mol. Cell. Biol., 2:1304-1319 (1982)) and glutamine synthetase (GS) amplification (See, for example, U.S. Pat. No. 5,122,464 and EP Patent Application 0 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2μ replication genes REP 1-3 and origin of replication.

The vector may also comprise a selectable marker, namely, a gene or polynucleotide, the product of which complements a defect in the host cell, such as the gene coding for dihydrofolate reductase (DHFR) or the Schizosaccharomyces pombe TPI gene (See, P. R. Russell, Gene, 40: 125-130 (1985)), or one which confers resistance to a drug, such as, ampicillin, kanamycin, tetracycline, chloramphenicol, neomycin, hygromycin or methotrexate. For filamentous fungi, selectable markers include, but are not limited to, amdS, pyrG, arcB, niaD and sC.

As used herein, the phrase “control sequences” refers to any components, which are necessary or advantageous for the expression of mammalian NGAL. Each control sequence may be native or foreign to the nucleic acid sequence encoding the mammalian NGAL. Such control sequences include, but are not limited to, a leader, polyadenylation sequence, propeptide sequence, promoter, enhancer or upstream activating sequence, signal peptide sequence and transcription terminator. At a minimum, the control sequences include at least one promoter operably linked to the polynucleotide sequence encoding the mammalian NGAL.

As used herein, the phrase “operably linked” refers to the covalent joining of two or more polynucleotide sequences, by means of enzymatic ligation or otherwise, in a configuration relative to one another such that the normal function of the sequences can be performed. For example, a polynucleotide sequence encoding a presequence or secretory leader is operably linked to a polynucleotide sequence for a polypeptide if it is expressed as a preprotein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the polynucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof. Examples of suitable control sequences for directing transcription in mammalian cells include the early and late promoters of SV40 and adenovirus, for example, the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus E1b region polyadenylation signals and the Kozak consensus sequence (Kozak, J Mol Biol., 196:947-50 (1987)).

In order to improve expression in mammalian cells a synthetic intron may be inserted in the 5′ untranslated region of the polynucleotide sequence encoding the mammalian NGAL. An example of a synthetic intron is the synthetic intron from the plasmid pCI-Neo (available from Promega Corporation, WI, USA).

Examples of suitable control sequences for directing transcription in insect cells include, but are not limited to, the polyhedrin promoter, the P10 promoter, the baculovirus immediate early gene 1 promoter and the baculovirus 39K delayed-early gene promoter and the SV40 polyadenylation sequence.

Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4-c promoter and the inducible GAL promoter.

Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulas glucoamylase, A. nidulans acetamidase, Rhizomucor miehei aspartic proteinase or lipase, the TPI1 terminator and the ADH3 terminator.

The polynucleotide sequence encoding the mammalian NGAL may or may not also include a polynucleotide sequence that encodes a signal peptide. The signal peptide is present when the mammalian NGAL is to be secreted from the cells in which it is expressed. Such signal peptide, if present, should be one recognized by the cell chosen for expression of the polypeptide. The signal peptide may be homologous (for example, it may be that normally associated with the mammalian NGAL of interest) or heterologous (namely, originating from another source than the mammalian NGAL of interest) to the mammalian NGAL of interest or may be homologous or heterologous to the host cell, namely, be a signal peptide normally expressed from the host cell or one which is not normally expressed from the host cell. Accordingly, the signal peptide may be prokaryotic, for example, derived from a bacterium, or eukaryotic, for example, derived from a mammalian, or insect, filamentous fungal or yeast cell.

The presence or absence of a signal peptide will, for example, depend on the expression host cell used for the production of the mammalian NGAL. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. For use in insect cells, the signal peptide may be derived from an insect gene (See, WO 90/05783), such as the lepidopteran Manduca sexta adipokinetic hormone precursor, (See, U.S. Pat. No. 5,023,328), the honeybee melittin (Invitrogen Corp., Carlsbad, Calif.), ecdysteroid UDP glucosyltransferase (egt) (Murphy et al., Protein Expression and Purification 4: 349-357 (1993), or human pancreatic lipase (hpl) (Methods in Enzymology, 284:262-272 (1997)).

Specific examples of signal peptides for use in mammalian cells include murine Ig kappa light chain signal peptide (Coloma, M, J. Imm. Methods, 152:89-104 (1992)). For use in yeast cells suitable signal peptides include the α-factor signal peptide from S. cerevisiae (See, U.S. Pat. No. 4,870,008), the signal peptide of mouse salivary amylase (See, O. Hagenbuchle et al., Nature, 289:643-646 (1981)), a modified carboxypeptidase signal peptide (See, L. A. Valls et al., Cell, 48:887-897 (1987)), the yeast BAR1 signal peptide (See, WO 87/02670), and the yeast aspartic protease 3 (YAP3) signal peptide (See, M. Egel-Mitani et al., Yeast, 6:127-137 (1990)).

Any suitable host may be used to produce the glycosylated mammalian NGAL of the present invention, including bacteria, fungi (including yeasts), plant, insect mammal or other appropriate animal cells or cell lines, as well as transgenic animals or plants. When a non-glycosylating organism such as E. coli is used, the expression in E. coli is preferably followed by suitable in vitro glycosylation in order to produce the glycosylated mammalian NGAL of the present invention.

Examples of bacterial host cells include, but are not limited to, gram positive bacteria such as strains of Bacillus, for example, B. brevis or B. subtilis, Pseudomonas or Streptomyces, or gram negative bacteria, such as strains of E. coli. The introduction of a vector into a bacterial host cell may, for instance, be effected by protoplast transformation (See, for example, Chang et al., Molecular General Genetics, 168:111-115 (1979)), using competent cells (See, for example, Young et al., Journal of Bacteriology, 81:823-829 (1961)), or Dubnau et al., Journal of Molecular Biology, 56:209-221 (1971)), electroporation (See, for example, Shigekawa et al., Biotechniques, 6:742-751 (1988)), or conjugation (See, for example, Koehler et al., Journal of Bacteriology, 169:5771-5278 (1987)).

Examples of suitable filamentous fungal host cells include, but are not limited to, strains of Aspergillus, for example, A. oryzae, A. niger, or A. nidulans, Fusarium or Trichoderma. Fungal cells may be transformed by a process involving protoplast formation, transformation of the protoplasts, and regeneration of the cell wall using techniques known to those skilled in the art. Suitable procedures for transformation of Aspergillus host cells are described in EP Patent Application 238 023 and U.S. Pat. No. 5,679,543. Suitable methods for transforming Fusarium species are described by Malardier et al., Gene, 78:147-156 (1989) and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al, Journal of Bacteriology, 153:163 (1983); and Hinnen et al., Proceedings of the National Academy of Sciences USA, 75:1920 (1978).

Preferably, the mammalian NGAL of the present invention is glycosylated in vivo. When the mammalian NGAL is to be glycosylated in vivo, the host cell is selected from a group of host cells capable of generating the desired glycosylation of the mammalian NGAL. Thus, the host cell may be selected from a yeast cell, insect cell, or mammalian cell.

Examples of suitable yeast host cells include strains of Saccharomyces, for example, S. cerevisiae, Schizosaccharomyces, Klyveromyces, Pichia, such as P. pastoris or P. methanolica, Hansenula, such as H. polymorpha or yarrowia. Methods for transforming yeast cells with heterologous polynucleotides and producing heterologous polypeptides therefrom are disclosed by Clontech Laboratories, Inc, Palo Alto, Calif., USA (in the product protocol for the Yeastmaker™ Yeast Tranformation System Kit), and by Reeves et al., FEMS Microbiology Letters, 99:193-198 (1992), Manivasakam et al., Nucleic Acids Research, 21:4414-4415 (1993) and Ganeva et al., FEMS Microbiology Letters, 121:159-164 (1994).

Examples of suitable insect host cells include, but are not limited to, a Lepidoptora cell line, such as Spodoptera frugiperda (Sf9 or Sf21) or Trichoplusia ni cells (High Five) (See, U.S. Pat. No. 5,077,214). Transformation of insect cells and production of heterologous polypeptides are well known to those skilled in the art.

Examples of suitable mammalian host cells include Chinese hamster ovary (CHO) cell lines, Green Monkey cell lines (COS), mouse cells (for example, NS/O), Baby Hamster Kidney (BHK) cell lines, human cells (such as, human embryonic kidney cells (for example, HEK293 (ATCC Accession No. CRL-1573))) and plant cells in tissue culture. Preferably, the mammalian host cells are CHO cell lines and HEK293 cell lines. Another preferred host cell is the B3 cell line (e.g., Abbott Laboratories, Abbott Bioresearch Center, Worcester, Mass.), or another dihydrofolate reductase deficient (DHFR⁻) CHO cell line (e.g., available from Invitrogen Corp., Carlsbad, Calif.). In one aspect, the present invention relates to a CHO cell line which produces glycosylated human wild-type NGAL (namely, that which has the amino acid sequence of SEQ ID NOS:1 or 12), wherein the CHO cell line has been deposited with American Type Culture Collection (ATCC) on Nov. 21, 2006 and received ATCC Accession No. PTA-8020. Preferably, the wild-type human NGAL produced by the CHO cell line having ATCC Accession No. PTA-8020 has a molecular weight of about 25 kilodaltons (kDa). In another aspect, the present invention relates to a CHO cell line which produces glycosylated mutant human NGAL. Preferably, the glycosylated mutant human NGAL comprises an amino acid substitution at the amino acid corresponding to amino acid 87 of the amino acid sequence of wild-type human NGAL (namely, SEQ ID NOS:1 or 12). More preferably, the amino acid substitution is the replacement of a cysteine with a serine (See, SEQ ID NOS:2 or 10). Most preferably, the CHO cell line is a CHO cell line that has been deposited with the ATCC on Jan. 23, 2007 and received ATCC Accession No. PTA-8168. The CHO cell line having ATCC Accession No. PTA-8168 produces a glycosylated mutant human NGAL comprising an amino acid sequence of SEQ ID NOS:2 or 10. In yet another aspect, the present invention relates to an isolated mutant glycosylated human NGAL comprising the amino acid sequence of SEQ ID NOS:2 or 10.

Methods for introducing exogenous polynucleotides into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamine™ 2000. These methods are well known in the art and are described, for example by Ausbel et al. (eds.) Current Protocols in Molecular Biology John Wiley & Sons, New York, USA (1996). The cultivation of mammalian cells are conducted according to established methods, e.g. as disclosed in Jenkins, Ed., Animal Cell Biotechnology, Methods and Protocols, Human Press Inc. Totowa, N.J., USA (1999) and Harrison and Rae General Techniques of Cell Culture, Cambridge University Press (1997).

In the production methods, cells are cultivated in a nutrient medium suitable for production of the mammalian NGAL using methods known in the art. For example, cells are cultivated by shake flask cultivation, small-scale or large-scale fermentation (including continuous, batch, fed-batch, or solid state fermentations) in laboratory or industrial fermenters performed in a suitable medium and under conditions allowing the glycosylated mammalian NGAL to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the glycosylated mammalian NGAL is secreted into the nutrient medium, the mammalian NGAL can be recovered directly from the medium. If the mammalian NGAL is not secreted, it can be recovered from cell lysates.

The resulting mammalian NGAL may be recovered by methods known in the art. For example, the mammalian NGAL may be recovered from the nutrient medium by conventional procedures including, but not limited to, centrifugation, filtration, extraction, spray drying, evaporation, or precipitation.

The mammalian NGAL may be purified by a variety of procedures known in the art including, but not limited to, chromatography (such as, but not limited to, ion exchange, affinity, hydrophobic, chromatofocusing, and size exclusion), electrophoretic procedures (such as, but not limited to, preparative isoelectric focusing), differential solubility (such as, but not limited to, ammonium sulfate precipitation), SDS-PAGE, or extraction (See, for example, J-C Janson and Lars Ryden, editors, Protein Purification, VCH Publishers, New York (1989)).

The glycosylated mammalian NGAL (wild-type and mutant) described herein can be used for a variety of different purposes and in a variety of different ways. Specifically, the glycosylated mammalian NGAL described herein can be used as one or more calibrators, one or more controls or as a combination of one or more calibrators or controls in an assay, preferably, an immunoassay, for detecting mammalian NGAL in a test sample. Preferably, the glycosylated mammalian NGAL comprises the amino acid sequence of SEQ ID NOS:1 or 12. Alternatively, the glycosylated mammalian NGAL comprises the amino acid sequence of SEQ ID NOS:2 or 10.

The use of glycosylated mammalian NGAL (wild-type and mutant) described herein as calibrators and controls in particular assays is described in U.S. Provisional Application Ser. No. 60/981,473 filed Oct. 19, 2007 (incorporated by reference for its teachings regarding same). The benefits in particular of use of the mutant NGAL (e.g., as set forth in SEQ ID NOS:2 or 10), optionally glycosylated are described in the examples of U.S. Provisional Application Ser. No. 60/981,473 filed Oct. 19, 2007 (incorporated by reference for its teachings regarding same). Namely, as set forth therein traditional NGAL assays detect monomers better than dimers, which will be perceived as a loss of monomers (i.e., as an instability) and will shift the calibration curve.

Furthermore, the mammalian NGAL according to the invention can be employed as immunogen to immunize animals for antibody production, e.g., where the animal can be a murine, rabbit, chicken, rat, sheep, goat, shark, camel, horse, feline, canine, non-human primate, human or other animal. In one embodiment, the immunogen comprises glycosylated mammalian NGAL, especially glycosylated human NGAL comprising the sequence of SEQ ID NO: 1, 2, 10 or 12. In another embodiment, the mammalian NGAL is that of a canine, feline, rat, mouse, horse, non-human primate, human, or other mammal.

### C. Method of Preventing Dimer Formation

In another aspect, the present invention relates to a method of preventing or eliminating the formation of at least one dimer of mammalian NGAL in a calibrator, control, or other sample (e.g., preferably test sample). The formation of the dimer to be prevented or eliminated can be mammalian NGAL homodimer or mammalian NGAL heterodimer.

The method of preventing or eliminating the formation of at least one dimer in a calibrator or control or other sample involves introducing at least one amino acid substitution into mammalian NGAL, preferably human NGAL having the amino acid sequence of SEQ ID NOS:1 or 12. Most preferably, the amino acid substitution involves the replacement of a cysteine with a serine at the amino acid corresponding to amino acid residue 87 of the wild-type sequence of human NGAL (namely, SEQ ID NOS:1 or 12). As a result of this amino acid substitution, the resulting human NGAL has the amino acid sequence of SEQ ID NOS:2 or 10. This mutant human NGAL, when added to a sample, such as a test sample, or when employed as a calibrator or control does not form any dimer (namely, homodimer or heterodimer), or forms a reduced amount of dimer (as compared to wild-type NGAL) with itself, with any wild-type human NGAL (SEQ ID NOS:1 or 12) present in the sample, or with any moiety capable of complexing with NGAL (e.g., gelatinase, MMP-9, others). Accordingly, the present invention further provides mutant mammalian NGAL that optionally is glycosylated. Such mutant mammalian NGAL optionally can be employed as a calibrator or control.

### D. Use in Making Human NGAL Antibodies

The human NGAL sequences of the present invention optionally can be employed to antibodies that specifically bind to wild-type human NGAL (namely, SEQ ID NOS:1 or 12) or human NGAL fragment, and that also optionally bind to human NGAL wherein the amino acid sequence contains at least one amino acid substitution of the wild-type sequence (SEQ ID NOS:1 or 12) so as to comprise a mutant or non-native sequence (e.g., SEQ ID NOS:2 or 10).

Antibodies directed against the polypeptides as described herein, and methods of making such antibodies using the polypeptides are described in U.S. Provisional Application Ser. No. 60/981,471 filed Oct. 19, 2007 (incorporated by reference for its teachings regarding same). Furthermore, the use of such antibodies as well as the polypeptides of the present invention, e.g., in immunoassays and/or as calibrators, controls, and immunodiagnostic agents, are described in U.S. Provisional Application Ser. No. 60/981,471 filed Oct. 19, 2007 (incorporated by reference for its teachings regarding same).

The antibodies can be made using a variety of different techniques known in the art. For example, polyclonal and monoclonal antibodies against wild-type human NGAL can be raised by immunizing a suitable subject (such as, but not limited to, a rabbit, goat, murine or other mammal) with an immunogenic preparation which contains a suitable immunogen. The immunogen that can be used for the immunization can include cells such as cells from immortalized cell lines NSO which is known to express human NGAL.

Alternatively, the immunogen can be the purified or isolated human wild-type NGAL protein itself (namely, SEQ ID NOS:1 or 12) or a human NGAL fragment thereof. For example, wild-type human NGAL (See, SEQ ID NOS:1 or 12) that has been isolated from a cell which produces the protein (such as NSO) using affinity chromatography, immunoprecipitation or other techniques which are well known in the art, can be used as an immunogen. Alternatively, immunogen can be prepared using chemical synthesis using routine techniques known in the art (such as, but not limited to, a synthesizer).

The antibodies raised in the subject can then be screened to determine if the antibodies bind to wild-type human NGAL or human NGAL fragment. Such antibodies can be further screened using the methods described in U.S. Provisional Application Ser. No. 60/981,471 filed Oct. 19, 2007 (incorporated by reference for its teachings regarding same). (See, e.g., Example 1). For example, these antibodies can be assayed to determine if they bind to amino acid residues 112, 118 and 147 of wild-type human NGAL or amino acid residues 15 and 109 of wild-type human NGAL (See, SEQ ID NOS:1 or 12). Suitable methods to identify an antibody with the desired characteristics are described therein (See, Example, 1). Moreover, it is fully anticipated that results obtained with antibodies that bind to mutant NGAL (See, SEQ ID NOS:2 or 10). are fully translatable to binding of wild-type NGAL, and that antibodies will bind to comparable residues of wild-type human NGAL (See, SEQ ID NOS:1 or 12). Accordingly, for convenience, and unless there lacks a rational basis in a particular instance for not doing so, mutant NGAL can be employed to assess binding properties of antibodies.

### E. Sample Collection and Pretreatment

Methods well known in the art for collecting, handling and processing urine, blood, serum and plasma, and other body fluids, are used in the practice of the present invention.

The test sample may comprise further moieties in addition to the NGAL analyte of interest such as antibodies, antigens, haptens, hormones, drugs, enzymes, receptors, proteins, peptides, polypeptides, oligonucleotides or polynucleotides. For example, the sample may be a whole blood sample obtained from a subject. It may be necessary or desired that a test sample, particularly whole blood, be treated prior to immunoassay as described herein, e.g., with a pretreatment reagent. Even in cases where pretreatment is not necessary (e.g., most urine samples), pretreatment optionally may be done for mere convenience (e.g., as part of a regimen on a commercial platform). The pretreatment reagent can be a heterogeneous agent or a homogeneous agent.

With use of a heterogenous pretreatment reagent according to the invention, the pretreatment reagent precipitates analyte binding protein (e.g., protein capable of binding NGAL) present in the sample. Such a pretreatment step comprises removing any analyte binding protein by separating from the precipitated analyte binding protein the supernatant of the mixture formed by addition of the pretreatment agent to sample. In such an assay, the supernatant of the mixture absent any binding protein is used in the assay, proceeding directly to the antibody capture step.

With use of a homogeneous pretreatment reagent there is no such separation step. The entire mixture of test sample and pretreatment reagent are contacted with the capture antibody in the antibody capture step. The pretreatment reagent employed for such an assay typically is diluted in the pretreated test sample mixture, either before the antibody capture step or during encounter with the antibody in the antibody capture step. Despite such dilution, a certain amount of the pretreatment reagent (for example, 5 M methanol and/or 0.6 M ethylene glycol) is still present (or remains) in the test sample mixture during antibody capture.

The pretreatment reagent can be any reagent appropriate for use with the immunoassay and kits of the invention. The pretreatment optionally comprises: (a) one or more solvents (e.g., methanol and ethylene glycol) and salt, (b) one or more solvents, salt and detergent, (c) detergent, or (d) detergent and salt. Pretreatment reagents are known in the art, and such pretreatment can be employed, e.g., as used for assays on Abbott TDx, AxSYM®, and ARCHITECT® analyzers (Abbott Laboratories, Abbott Park, Ill.), as described in the literature (see, e.g., Yatscoff et al., Abbott TDx Monoclonal Antibody Assay Evaluated for Measuring Cyclosporine in Whole Blood, Clin. Chem., 36:1969-1973 (1990) and Wallemacq et al., Evaluation of the New AxSYM Cyclosporine Assay: Comparison with TDx Monoclonal Whole Blood and EMIT Cyclosporine Assays, Clin. Chem. 45: 432-435 (1999)), and/or as commercially available. Additionally, pretreatment can be done as described in Abbott's U.S. Pat. No. 5,135,875, EP 0 471 293, U.S. Patent Application 60/878,017 filed Dec. 29, 2006; and U.S. patent application Ser. No. 11/490,624 filed Jun. 21, 2006 (incorporated by reference in its entirety for its teachings regarding pretreatment). Also, proteases, either alone or in combination with any other pretreament agents (e.g., solvents, detergents, salts, and the like) can be employed.

### F. NGAL Immunoassays

As previously discussed, the NGAL polypeptides, and antibodies obtained using the NGAL polypeptides, can be employed in immunoassays. Particular improved immunoassays can be conducted as described in U.S. Provisional Application Ser. No. 60/981,473 filed Oct. 19, 2007 (incorporated by reference for its teachings regarding same). However, NGAL immunoassays generally can be conducted using any format known in the art, such as, but not limited to, a sandwich format, as further described in U.S. Provisional Application Ser. No. 60/981,473.

Specifically, in one aspect of the present invention, at least two antibodies are employed to separate and quantify human NGAL or human NGAL fragment in a test sample. More specifically, the at least two antibodies bind to certain epitopes of human NGAL or human NGAL fragment forming an immune complex which is referred to as a “sandwich”. Generally, in the immunoassays one or more antibodies can be used to capture the human NGAL or human NGAL fragment in the test sample (these antibodies are frequently referred to as a “capture” antibody or “capture” antibodies) and one or more antibodies can be used to bind a detectable (namely, quantifiable) label to the sandwich (these antibodies are frequently referred to as the “detection antibody”, “detection antibodies”, a “conjugate” or “conjugates”).

Excellent immunoassays, particularly, sandwich assays, can be performed using the antibodies directed against the NGAL polypeptides of the present invention as the capture antibodies, detection antibodies or as capture and detection antibodies. These are described in detail in U.S. Provisional Application Ser. No. 60/981,473 filed Oct. 19, 2007 (incorporated by reference for its teachings regarding same).

Generally speaking, a test sample being tested for (for example, suspected of containing) human NGAL or human NGAL fragment can be contacted with at least one capture antibody (or antibodies) and at least one detection antibody (which is either a second detection antibody or a third detection antibody) either simultaneously or sequentially and in any order. For example, the test sample can be first contacted with at least one capture antibody and then (sequentially) with at least one detection antibody. Alternatively, the test sample can be first contacted with at least one detection antibody and then (sequentially) with at least one capture antibody. In yet another alternative, the test sample can be contacted simultaneously with a capture antibody and a detection antibody.

In the sandwich assay format, a test sample suspected of containing human NGAL or human NGAL fragment is first brought into contact with an at least one first capture antibody under conditions which allow the formation of a first antibody/human NGAL complex. If more than one capture antibody is used, a first multiple capture antibody/human NGAL complex is formed. In a sandwich assay, the antibodies, preferably, the at least one capture antibody, are used in molar excess amounts of the maximum amount of human NGAL or human NGAL fragment expected in the test sample. For example, from about 5 μg/mL to about 1 mg/mL of antibody per mL of buffer (e.g., microparticle coating buffer) can be used.

Optionally, prior to contacting the test sample with the at least one capture antibody (for example, the first capture antibody), the at least one capture antibody can be bound to a solid support which facilitates the separation the first antibody/human NGAL complex from the test sample. Any solid support known in the art can be used, including, but not limited to, solid supports made out of polymeric materials in the forms of wells, tubes or beads. The antibody (or antibodies) can be bound to the solid support by adsorption, by covalent bonding using a chemical coupling agent or by other means known in the art, provided that such binding does not interfere with the ability of the antibody to bind human NGAL or human NGAL fragment. Alternatively, the antibody (or antibodies) can be bound with microparticles that have previously coated with streptavidin or biotin (for example, using Power-Bind™-SA-MP streptavidin coated microparticles, available from Seradyn, Indianapolis, Ind.). Alternatively, the antibody (or antibodies) can be bound using microparticles that have been previously coated with anti-species specific monoclonal antibodies. Moreover, if necessary, the solid support can be derivatized to allow reactivity with various functional groups on the antibody. Such derivatization requires the use of certain coupling agents such as, but not limited to, maleic anhydride, N-hydroxysuccinimide and 1-ethyl-3-(3-dimethylaminopropyl)carbodiimide.

After the test sample being tested for and/or suspected of containing human NGAL or a human NGAL fragment is brought into contact with the at least one capture antibody (for example, the first capture antibody), the mixture is incubated in order to allow for the formation of a first antibody (or multiple antibody)-human NGAL complex. The incubation can be carried out at a pH of from about 4.5 to about 10.0, at a temperature of from about 2° C. to about 45° C., and for a period from at least about one (1) minute to about eighteen (18) hours, preferably from about 1 to about 20 minutes, most preferably for about 18 minutes. The immunoassay described herein can be conducted in one step (meaning the test sample, at least one capture antibody and at least one detection antibody are all added sequentially or simultaneously to a reaction vessel) or in more than one step, such as two steps, three steps, etc.

After formation of the (first or multiple) capture antibody/human NGAL complex, the complex is then contacted with at least one detection antibody (under conditions which allow for the formation of a (first or multiple) capture antibody/human NGAL/second antibody detection complex). The at least one detection antibody can be the second, third, fourth, etc. antibodies used in the immunoassay. If the capture antibody/human NGAL complex is contacted with more than one detection antibody, then a (first or multiple) capture antibody/human NGAL/(multiple) detection antibody complex is formed. As with the capture antibody (e.g., the first capture antibody), when the at least second (and subsequent) detection antibody is brought into contact with the capture antibody/human NGAL complex, a period of incubation under conditions similar to those described above is required for the formation of the (first or multiple) capture antibody/human NGAL/(second or multiple) detection antibody complex. Preferably, at least one detection antibody contains a detectable label. The detectable label can be bound to the at least one detection antibody (e.g., the second detection antibody) prior to, simultaneously with or after the formation of the (first or multiple) capture antibody/human NGAL/(second or multiple) detection antibody complex. Any detectable label known in the art can be used. For example, the detectable label can be a radioactive label, such as, ³H, ¹²⁵I, ³⁵S, ¹⁴C, ³²P, ³³P, an enzymatic label, such as horseradish peroxidase, alkaline phosphatase, glucose 6-phosphate dehydrogenase, etc., a chemiluminescent label, such as, acridinium esters, luminal, isoluminol, thioesters, sulfonamides, phenanthridinium esters, etc. a fluorescence label, such as, fluorescein (5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, etc.), rhodamine, phycobiliproteins, R-phycoerythrin, quantum dots (zinc sulfide-capped cadmium selenide), a thermometric label or an immuno-polymerase chain reaction label. An introduction to labels, labeling procedures and detection of labels is found in Polak and Van Noorden, Introduction to Immunocytochemistry, 2^{nd} ed., Springer Verlag, N.Y. (1997) and in Haugland, Handbook of Fluorescent Probes and Research Chemicals (1996), which is a combined handbook and catalogue published by Molecular Probes, Inc., Eugene, Oreg.

The detectable label can be bound to the antibodies either directly or through a coupling agent. An example of a coupling agent that can be used is EDAC (1-ethyl-3-(3-dimethylaminopropyl) carbodiimide, hydrochloride) that is commercially available from Sigma-Aldrich, St. Louis, Mo. Other coupling agents that can be used are known in the art. Methods for binding a detectable label to an antibody are known in the art. Additionally, many detectable labels can be purchased or synthesized that already contain end groups that facilitate the coupling of the detectable label to the antibody, such as, N10-(3-sulfopropyl)-N-(3-carboxypropyl)-acridinium-9-carboxamide, otherwise known as CPSP-Acridinium Ester or N10-(3-sulfopropyl)-N-(3-sulfopropyl)-acridinium-9-carboxamide, otherwise known as SPSP-Acridinium Ester.

The (first or multiple) capture antibody/human NGAL/(second or multiple) detection antibody complex can be, but does not have to be, separated from the remainder of the test sample prior to quantification of the label. For example, if the at least one capture antibody (e.g., the first capture antibody) is bound to a solid support, such as a well or a bead, separation can be accomplished by removing the fluid (of the test sample) from contact with the solid support. Alternatively, if the at least first capture antibody is bound to a solid support it can be simultaneously contacted with the human NGAL-containing sample and the at least one second detection antibody to form a first (multiple) antibody/human NGAL/second (multiple) antibody complex, followed by removal of the fluid (test sample) from contact with the solid support. If the at least one first capture antibody is not bound to a solid support, then the (first or multiple) capture antibody/human NGAL/(second or multiple) detection antibody complex does not have to be removed from the test sample for quantification of the amount of the label.

After formation of the labeled capture antibody/human NGAL/detection antibody complex (e.g., the first capture antibody/human NGAL/second detection antibody complex), the amount of label in the complex is quantified using techniques known in the art. For example, if an enzymatic label is used, the labeled complex is reacted with a substrate for the label that gives a quantifiable reaction such as the development of color. If the label is a radioactive label, the label is quantified using a scintillation counter. If the label is a fluorescent label, the label is quantified by stimulating the label with a light of one color (which is known as the “excitation wavelength”) and detecting another color (which is known as the “emission wavelength”) that is emitted by the label in response to the stimulation. If the label is a chemiluminescent label, the label is quantified detecting the light emitted either visually or by using luminometers, x-ray film, high speed photographic film, a CCD camera, etc. Once the amount of the label in the complex has been quantified, the concentration of human NGAL or human NGAL fragment in the test sample is determined by use of a standard curve that has been generated using serial dilutions of human NGAL or human NGAL fragment of known concentration. Other than using serial dilutions of human NGAL or human NGAL fragment, the standard curve can be generated gravimetrically, by mass spectroscopy and by other techniques known in the art.

The methods described herein (namely, the immunoassays and kits) can be used to evaluate the renal tubular cell injury status of a subject based on the determination of the level of NGAL present in the test sample. The subject to be evaluated can either currently have renal tubular cell injury or be at risk of developing renal tubular cell injury.

The methods described herein can be carried out on a subject after treatment of a subject for renal tubular cell injury or while the subject is currently experiencing renal tubular cell injury.

The methods described herein can be used to monitor the nephrotoxic side effects of drugs or other therapeutic agents in a subject.

The methods described herein can be carried out or performed after an event experienced by a subject, such as after a surgical procedure (such as after cardiac surgery, coronary bypass surgery, cardiovascular surgery, vascular surgery or kidney transplantation), after the subject has experienced a diminished blood supply to the kidneys, if the subject has or is experiencing a medical condition selected from the group consisting of: impaired heart function, stroke, trauma, sepsis and dehydration, admittance of a subject to an intensive care unit, after administration to the subject of one or more pharmaceuticals, or after administration to the subject of one or more contrast agents.

It goes without saying that while certain embodiments herein are advantageous when employed to assess renal tubular cell injury status, the immunoassays and kits also optionally can be employed to assess NGAL in other diseases, e.g., cancer, sepsis, and any disease disorder or condition involving assessment of NGAL.

More specifically, in addition to assessment of renal disorders, diseases and injuries (see, e.g., U.S. Pat. App. Pub. Nos. 2008/0090304, 2008/0014644, 2008/0014604, 2007/0254370, and 2007/0037232), the assay and assay components as described herein optionally can also be employed in any other NGAL assay or in any other circumstance in which an assessment of NGAL levels or concentration might prove helpful: e.g., cancer-related assays (e.g., generally, or more specifically including but not limited to pancreatic cancer, breast cancer, ovarian/uterine cancer, leukemia, colon cancer, and brain cancer; see, e.g., U.S. Pat. App. Pub. No. 2007/0196876; see, also, U.S. Pat. Nos. 5,627,034 and 5,846,739); diagnosis of systemic inflammatory response syndrome (SIRS), sepsis, severe sepsis, septic shock and multiple organ dysfunction syndrome (MODS) (see, e.g., U.S. Pat. App. Pub. Nos. 2008/0050832 and 2007/0092911; see, also, U.S. Pat. No. 6,136,526); hematology applications (e.g., estimation of cell type); assessment of preeclampsia, obesity (metabolic syndrome), insulin resistance, hyperglycemia, tissue remodeling (when complexed with MMP-9; see, e.g., U.S. Pat. App. Pub. No. 2007/0105166 and U.S. Pat. No. 7,153,660), autoimmune diseases (e.g., rheumatoid arthritis, inflammatory bowel disease, multiple sclerosis), irritable bowel syndrome (see, e.g., U.S. Pat. App. Pub. Nos. 2008/0166719 and 2008/0085524), neurodegenerative disease, respiratory tract disease, inflammation, infection, periodontal disease (see, e.g., U.S. Pat. No. 5,866,432), and cardiovascular disease including venous thromboembolic disease (see, e.g., U.S. Pat. App. Pub. Nos. 2007/0269836), among others.

### G. NGAL Immunoassay Kits

The present invention also contemplates kits for detecting the presence of mammalian NGAL antigen in a test sample. Optionally, the kit can also contain at least one calibrator or control as described herein. For the other NGAL assays (e.g., those described in U.S. Provisional Application Ser. No. 60/981,473 filed Oct. 19, 2007 (incorporated by reference for its teachings regarding same), potentially other calibrators or controls can be included in an NGAL immunoassay kit. Preferably, however, for an immunoassay kit as described herein, the calibrator or control is mammalian NGAL, especially glycosylated human NGAL (e.g., wild-type or mutant) as set forth herein.

Accordingly, the kits of the invention can comprise at least one calibrator, or at least one control, or a combination of at least one calibrator and at least one control, wherein the calibrator or control comprises a glycosylated mammalian NGAL of the present invention. Preferably, the at least one calibrator or at least one control is a glycosylated mammalian NGAL having the amino acid sequence selected from the group consisting of: SEQ ID NO:1, SEQ ID NO:2, SEQ ID NO:10, SEQ ID NO:12, and combinations of SEQ ID NOS:1, 2, 10 or 12. If the kit is a kit for performing an immunoassay, then the kit optionally further comprises: (1) at least one capture antibody that specifically binds to mammalian NGAL; (2) at least one conjugate; (3) one or more instructions for performing the immunoassay; or (4) or any combination of items (1)-(3).

Thus, the present invention further provides for diagnostic and quality control kits comprising mammalian NGAL of the invention. Optionally the assays, kits and kit components of the invention are optimized for use on commercial platforms (e.g., immunoassays on the Prism®, AxSYM®, ARCHITECT® and EIA (Bead) platforms of Abbott Laboratories, Abbott Park, Ill., as well as other commercial and/or in vitro diagnostic assays). Additionally, the assays, kits and kit components can be employed in other formats, for example, on electrochemical or other hand-held or point-of-care assay systems. The present invention is, for example, applicable to the commercial Abbott Point of Care (i-STAT®, Abbott Laboratories, Abbott Park, Ill.) electrochemical immunoassay system that performs sandwich immunoassays for several cardiac markers, including TnI, CKMB and BNP. Immunosensors and methods of operating them in single-use test devices are described, for example, in US Patent Applications 20030170881, 20040018577, 20050054078 and 20060160164 which are incorporated herein by reference. Additional background on the manufacture of electrochemical and other types of immunosensors is found in U.S. Pat. No. 5,063,081 which is also incorporated by reference for its teachings regarding same.

Optionally the kits include quality control reagents (e.g., sensitivity panels, calibrators, and positive controls). Preparation of quality control reagents is well known in the art, and is described, e.g., on a variety of immunodiagnostic product insert sheets. NGAL sensitivity panel members optionally can be prepared in varying amounts containing, e.g., known quantities of NGAL antigen ranging from “low” to “high”, e.g., by spiking known quantities of the NGAL antigen according to the invention into an appropriate assay buffer (e.g., a phosphate buffer). These sensitivity panel members optionally are used to establish assay performance characteristics, and further optionally are useful indicators of the integrity of the immunoassay kit reagents, and the standardization of assays.

In another embodiment, the present invention provides for a quality control kit comprising one or more antigens of the present invention for use as a sensitivity panel to evaluate assay performance characteristics and/or to quantitate and monitor the integrity of the antigen(s) used in the assay.

In still another embodiment, the mammalian NGAL (e.g., glycosylated mammalian NGAL) according to the invention can be employed as calibrators and/or controls. The antibodies provided in the kit can incorporate a detectable label, such as a fluorophore, radioactive moiety, enzyme, biotin/avidin label, chromophore, chemiluminescent label, or the like, or the kit may include reagents for labeling the antibodies or reagents for detecting the antibodies (e.g., detection antibodies) and/or for labeling the antigens or reagents for detecting the antigen. The antibodies, calibrators and/or controls can be provided in separate containers or pre-dispensed into an appropriate assay format, for example, into microtiter plates.

The kits can optionally include other reagents required to conduct a diagnostic assay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), may also be included in the kit. The kit may additionally include one or more other controls. One or more of the components of the kit may be lyophilized and the kit may further comprise reagents suitable for the reconstitution of the lyophilized components.

The various components of the kit optionally are provided in suitable containers. As indicated above, one or more of the containers may be a microtiter plate. The kit further can include containers for holding or storing a sample (e.g., a container or cartridge for a blood or urine sample). Where appropriate, the kit may also optionally contain reaction vessels, mixing vessels and other components that facilitate the preparation of reagents or the test sample. The kit may also include one or more instruments for assisting with obtaining a test sample, such as a syringe, pipette, forceps, measured spoon, or the like.

The kit further can optionally include instructions for use, which may be provided in paper form or in computer-readable form, such as a disc, CD, DVD or the like.

By way of example, and not of limitation, examples of the present invention shall now be given.

### H. Adaptation of Assay Kit

The kit (or components thereof), as well as the method of determining the concentration of NGAL antigen in a test sample by an assay using the components described herein, can be adapted for use in a variety of automated and semi-automated systems (including those wherein the solid phase comprises a microparticle), as described, e.g., in U.S. Pat. Nos. 5,089,424 and 5,006,309, and as commercially marketed, e.g., by Abbott Laboratories (Abbott Park, Ill.) as ARCHITECT®.

Some of the differences between an automated or semi-automated system as compared to a non-automated system (e.g., ELISA) include the substrate to which the first specific binding partner (e.g., NGAL capture antibody) is attached (which can impact sandwich formation and analyte reactivity), and the length and timing of the capture, detection and/or any optional wash steps. Whereas a non-automated format such as an ELISA may require a relatively longer incubation time with sample and capture reagent (e.g., about 2 hours) an automated or semi-automated format (e.g., ARCHITECT®, Abbott Laboratories) may have a relatively shorter incubation time (e.g., approximately 18 minutes for ARCHITECT®). Similarly, whereas a non-automated format such as an ELISA may incubate a detection antibody such as the conjugate reagent for a relatively longer incubation time (e.g., about 2 hours), an automated or semi-automated format (e.g., ARCHITECT®) may have a relatively shorter incubation time (e.g., approximately 4 minutes for the ARCHITECT®).

Other platforms available from Abbott Laboratories include, but are not limited to, AxSYM®, IMx® (see, e.g., U.S. Pat. No. 5,294,404, which is hereby incorporated by reference in its entirety), PRISM®, EIA (bead), and Quantum™ II, as well as other platforms. Additionally, the assays, kits and kit components can be employed in other formats, for example, on electrochemical or other hand-held or point-of-care assay systems. The present disclosure is, for example, applicable to the commercial Abbott Point of Care (i-STAT®, Abbott Laboratories) electrochemical immunoassay system that performs sandwich immunoassays. Immunosensors and their methods of manufacture and operation in single-use test devices are described, for example in, U.S. Pat. No. 5,063,081, U.S. Pat. App. Pub. No. 2003/0170881, U.S. Pat. App. Pub. No. 2004/0018577, U.S. Pat. App. Pub. No. 2005/0054078, and U.S. Pat. App. Pub. No. 2006/0160164, which are incorporated in their entireties by reference for their teachings regarding same.

In particular, with regard to the adaptation of an NGAL assay to the I-STAT® system, the following configuration is preferred. A microfabricated silicon chip is manufactured with a pair of gold amperometric working electrodes and a silver-silver chloride reference electrode. On one of the working electrodes, polystyrene beads (0.2 mm diameter) with immobilized capture antibody are adhered to a polymer coating of patterned polyvinyl alcohol over the electrode. This chip is assembled into an I-STAT® cartridge with a fluidics format suitable for immunoassay. On a portion of the wall of the sample-holding chamber of the cartridge there is a layer comprising the second detection antibody labeled with alkaline phosphatase (or other label). Within the fluid pouch of the cartridge is an aqueous reagent that includes p-aminophenol phosphate.

In operation, a sample suspected of containing NGAL antigen is added to the holding chamber of the test cartridge and the cartridge is inserted into the I-STAT® reader. After the second antibody (detection antibody) has dissolved into the sample, a pump element within the cartridge forces the sample into a conduit containing the chip. Here it is oscillated to promote formation of the sandwich between NGAL antigen, NGAL capture antibody, and the labeled detection antibody. In the penultimate step of the assay, fluid is forced out of the pouch and into the conduit to wash the sample off the chip and into a waste chamber. In the final step of the assay, the alkaline phosphatase label reacts with p-aminophenol phosphate to cleave the phosphate group and permit the liberated p-aminophenol to be electrochemically oxidized at the working electrode. Based on the measured current, the reader is able to calculate the amount of NGAL antigen in the sample by means of an embedded algorithm and factory-determined calibration curve.

It further goes without saying that the methods and kits as described herein necessarily encompass other reagents and methods for carrying out the immunoassay. For instance, encompassed are various buffers such as are known in the art and/or which can be readily prepared or optimized to be employed, e.g., for washing, as a conjugate diluent, and/or as a calibrator diluent. An exemplary conjugate diluent is ARCHITECT® conjugate diluent employed in certain kits (Abbott Laboratories, Abbott Park, Ill.) and containing 2-(N-morpholino)ethanesulfonic acid (MES), a salt, a protein blocker, an antimicrobial agent, and a detergent. An exemplary calibrator diluent is ARCHITECT® human calibrator diluent employed in certain kits (Abbott Laboratories, Abbott Park, Ill.), which comprises a buffer containing MES, other salt, a protein blocker, and an antimicrobial agent.

### Example 1

### Human NGAL Wild-Type Antigen

### Human NGAL Wild-Type Gene Cloning

Human NGAL (LCN2, Homo sapien lipocalin-2, oncogene 24p3) plasmid clone pCMV6-XL4-NGAL (lipocalin-2, LCN2) (Origene Technologies Inc., Rockville, Md., Catalog number TC116655, NM_{—}005564.2) was used as template. A pair of PCR primers was designed to clone out the human (wild-type) NGAL gene. The 5′-end primer contained a partial NGAL signal sequence and a Srf I restriction site, and the 3′-end primer contained a Not I restriction site, 6×His and partial C-terminal NGAL sequence. The 5′ and 3-end primers are shown below.

NGAL 5′-end primer (N-forsrf):
(SEQ ID NO: 5)
5′-CTT GCC CGG GCG CAC CAT GCC CCT AGG TCT CCT
G-3′.
NGAL 3′-end primer (N-revhis):
(SEQ ID NO: 6)
5′-CCC CGC GGC CGC TCA ATG GTG ATG GTG ATG ATG
GCC GTC GAT ACA CTG GTC GAT TGG-3′

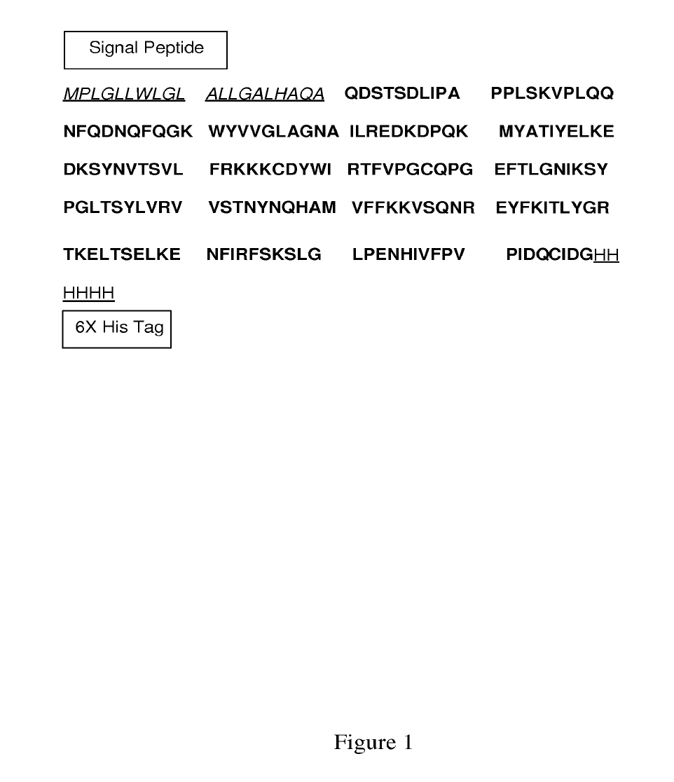

The PCR reaction was executed in 2× reaction Buffer (dNTP), with the 5′ and 3′ primers and 1.25 units of Pfx DNA polymerase (Invitrogen Corp., Carlsbad, Calif.). The PCR was performed for 30 cycles of 15 seconds at 94° C. followed by 1 min at 68° C. A total of 30 cycles were performed. The wild-type human NGAL antigen sequence including the signal peptide is shown in FIG. 1 (SEQ ID NO:1), and lacking the signal peptide is set forth in SEQ ID NO:12.

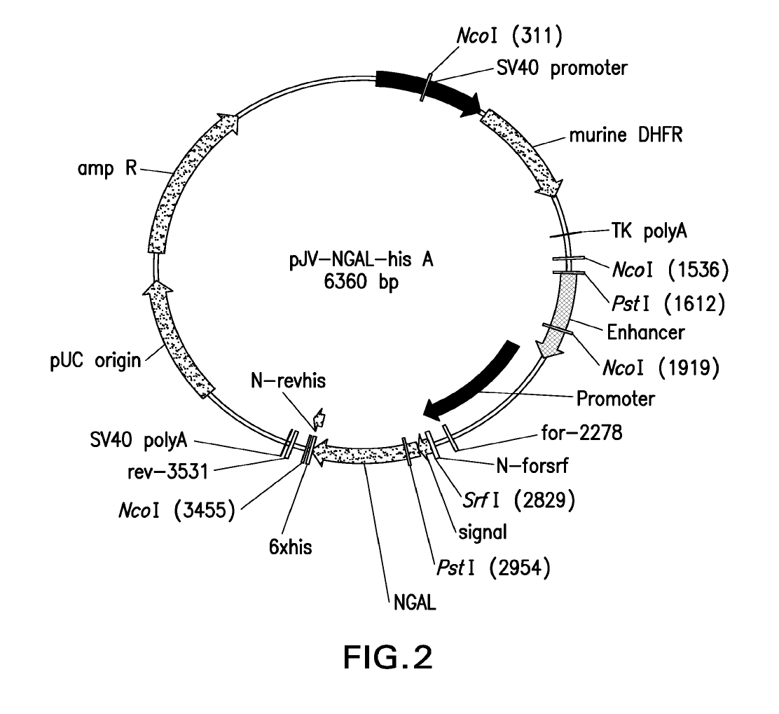

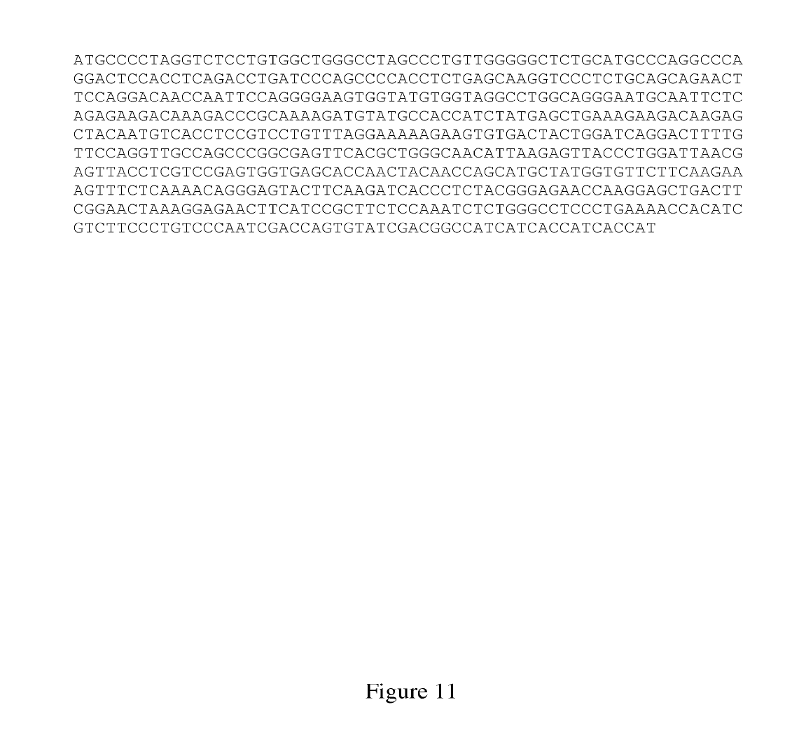

A 643 bp PCR product was gel purified and restriction enzyme trimmed by Srf and Not I, and then cloned into a pJV vector and transformed into E. coli DH5α. The pJV vector was obtained from Abbott Laboratories (Abbott Bioresearch Center, Worcester, Mass.) and comprises the ampicillin resistance gene, pUC origin, SV40 origin, EF-1a promoter. The resulting pJV-based vector, pJV-NGAL-HisA (also known as pJV-NGAL-A3, See FIG. 2) further comprises the full length wild-type human NGAL antigen sequence (See FIG. 11; also, encoding sequence set out at SEQ ID NO:3) including the human NGAL signal peptide sequence (shown in FIG. 1).

The transformed E. coli clones were grown in LB broth overnight with shaking at 37° C. Plasmid DNA was purified from each individual clone with the QIAprep spin miniprep kit (QIAGEN, Valencia, Calif.) followed by sequencing using the BigDye Terminator v3.1 Cycle Sequencing kit (Applied Biosystems, Foster City, Calif.). Plasmid pJV-NGAL-A3 (See FIG. 2) was selected by sequencing and analyzed by Vector NTI Advance™ software (Invitrogen Corp., Carlsbad, Calif.). Once the pJV clone was identified, separate E. coli DH5α cell banks containing pJV-NGAL-A3 plasmid were made to preserve the pJV clones.

### Human NGAL Wild-Type Antigen Transient Expression in HEK293 Cells

pJV-NGAL-A3 plasmid DNA was Maxi prepared using an Endofree plasmid Maxi kit (QIAGEN, Valencia, Calif.) according to standard techniques. The high purity plasmid DNA thus obtained was then transiently transfected into HEK293 cells by fectin (Invitrogen Corp., Carlsbad, Calif.). The transfected HEK293 cells were incubated at 37° C. in an 8% CO₂ incubator for three days, then harvested by centrifugation at 4000 rpm for 20 minutes. The supernatant was collected where the NGAL was secreted. The supernatant was dia-filtrated using Pelicon 2 mini (Millipore, Billerica, Mass.) three times to change the buffer to Phosphate Buffered Saline (PBS), pH 7.2.

### Human NGAL Wild-Type Antigen Purification

The dia-filtrated NGAL solution was purified using nickel-nitrilotriacetic acid (Ni-NTA, QIAGEN, Valencia, Calif.) metal-affinity chromatography. The NI-NTA superflow resin was assembled into a FPLC column, washed with 3 volumes of distilled water and pre-equilibrated with wash buffer (50 mM NaH₂PO₄, 10 mM imidazole, 300 mM NaCl, 0.05% Tween 20 and adjust the pH to 8.0 with 6N NaOH). The dia-filtrated NGAL sample was loaded onto the column at flow rate of 0.5 mL/min, washed with 10-20 volumes of wash buffer, and the NGAL protein was eluted from the column with elution buffer (50 mM NaH₂PO₄, 250 mM imidazole, 300 mM NaCl, 0.05% Tween 20 and the pH adjusted to 8.0 with 6N NaOH). Purified NGAL protein was dialyzed three times using 3-5 liters Phosphate Buffered Saline (PBS), pH 7.2.

### Establishing a Stable CHO Cell Line and Expression of the Human NGAL

A Chinese Hamster Ovary (CHO) cell line (B3.2, Abbott Laboratories, Abbott Bioresearch Center, Worcester, Mass.) that lacks the dihydrofolate reductase (DHFR) gene was used for transfection and stable human NGAL expression as described below. The CHO cells were cultured and transfected by standard calcium phosphate mediated transfection with the pJV-NGAL-A3 plasmid. The NGAL transfected CHO cells were selected for several weeks in alpha MEM medium (Invitrogen Corp., Carlsbad, Calif.) lacking ribonucleosides and deoxyribonucleosides, and containing 5% dialyzed FBS (dFBS) in 96-well plates. Once the CHO clones had grown to more than 50% confluency, the supernatant was tested by enzyme immunoassay (ETA) to rank the performance of the CHO clones. The supernatants from 96-well transfected CHO cells were coated on 96-well EIA plates for at least 1 hour at room temperature, and then were blocked with 2% BSA/PBS buffer for 1 hour. The murine anti-His monoclonal antibody was added into the coated wells and the plates were incubated for at least 1 hour at room temperature. After incubation, the plates were washed and incubated with horseradish peroxidase (HRP) labeled goat-anti mouse IgG antibody for about 1 hour. The plates were developed using O-Phenylenediamine-2HCl (OPD) and read at an optical density of 492 nm. The 10 CHO clones that gave the highest signal in the EIA were expanded and re-assayed. The EIA re-assay was executed with anti-NGAL mouse monoclonal antibody coated on the EIA 96-well plate, washed and then incubated with supernatant from cultured CHO cell clones, then washed again and incubated with anti-His (C-term)-HRP (Invitrogen Corp., Carlsbad, Calif.) and finally developed by using OPD as described above. A clone referred to as “CHO clone #204” was then selected based on the highest signal given in the EIA re-assay, and methotrexate (MTX) amplification was done to boost NGAL secretion.

### Methotrexate Amplification of CHO Cell Clone #204

20 nM MTX amplification: The NGAL recombinant antigen (rAg) CHO cell clone #204 described above was subcloned into alpha MEM medium +5% dFBS+20 nM MTX using end point dilution. After a few weeks amplification in MTX, ten CHO subclones were identified including CHO clone number 204-465 (also referred to as “CHO clone #465”). Identification was done using EIA employing anti-NGAL monoclonal antibody coated on EIA plates, washing and then incubating with supernatant from cultured CHO cell clones, then washing again and incubating with anti-His (C-term)-HRP (Invitrogen Corp., Carlsbad, Calif.), and finally, developing by OPD described as above. The human NGAL rAg CHO cell clone number 204-465 was selected based on the assay results for further MTX amplification.

100 nM MTX amplification: The human NGAL rAg CHO cell clone number 204-465 isolated as described as above was subcloned by end point dilution in 100 nM MTX supplemented into alpha MEM±5% dFBS medium in 96-well plates. After a few weeks amplification in MTX, 36 CHO subclones were initially identified including CHO clone number 204-465-950 using EIA as described above. A human NGAL rAg CHO cell clone number 204-465-950 (also referred to as “CHO clone #950”) was selected based on assay results for further MTX amplification.

500 nM MTX amplification: The human NGAL rAg CHO cell clone number 204-465-950 isolated as described as above was subcloned by end point dilution in 500 nM MTX supplemented into alpha MEM+5% dFBS medium in 96-well plates. After a few weeks amplification in MTX, CHO subclones were initially identified including CHO clone number 204-465-950-113 using EIA as described above. A human NGAL rAg CHO cell clone number 204-465-950-113 (also referred to as “CHO clone #113”) was selected based on assay results for further MTX amplification.

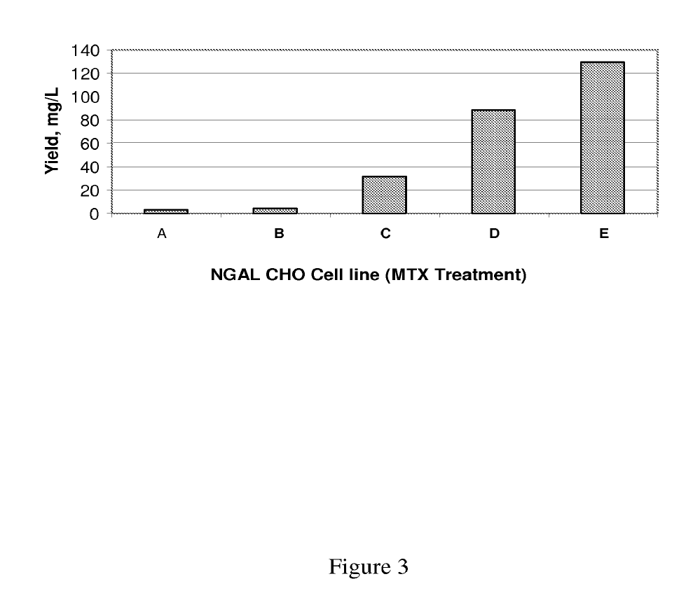

5 μM MTX amplification: In addition to the 500 nM amplification, the human NGAL rAg CHO cell clone number 204-465-950 went through a series of MTX amplifications in 2 μM and 5 μM MTX in a T flask. After a few weeks amplification, MTX amplified CHO cells were subcloned by end point dilution in 5 μM MTX supplemented into alpha MEM+5% dFBS medium in 96-well plates. 24 CHO subclones were initially identified including CHO clone number 204-465-950-662 (also referred to as “CHO clone #662”) using EIA as described as above. A human NGAL rAg CHO cell clone number 204-465-950-662 was selected and weaned into serum-free DHFR-CHO medium (Sigma-Aldrich, St. Louis, Mo.). A cell bank of CHO subclone number 204-465-950-662 was prepared and named NGAL rAg CHO 662. Human NGAL wild-type antigen production yields for subclones including CHO clone amplified by MTX are shown in FIG. 3, which varies from 3.8 mg/L to 129 mg/L.

### Example 2

### Characterization of Recombinant NGAL Antigen

### SDS-PAGE Gel Electrophoresis

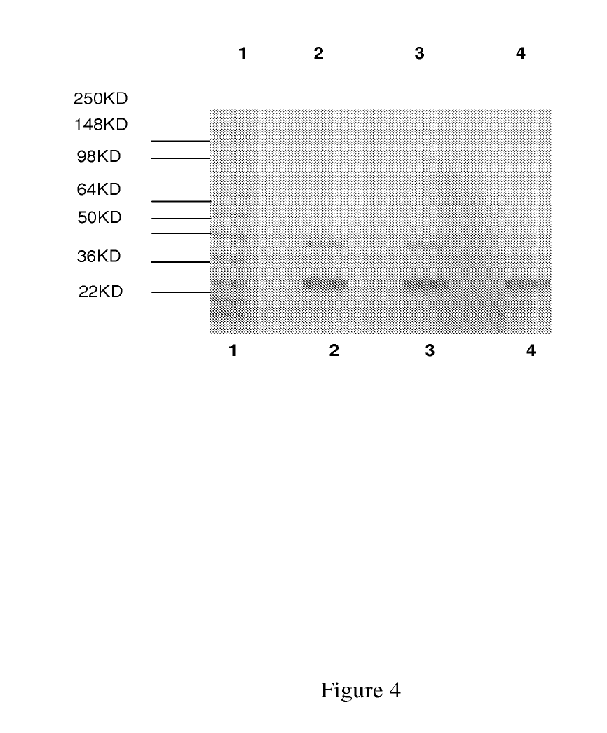

SDS-PAGE gel electrophoresis was performed on CHO cells (CHO clone #662) expressing human NGAL recombinant antigen under reducing condition or non-reducing conditions. About 4.5 μg of recombinant NGAL antigen was mixed with loading buffer with or without reducing agents (β-mercaptoethanol), boiled for 10 minutes, then loaded onto a 4-20% SDS-PAGE gel and run at 80 Volts for 1.5 hours. Monomer human NGAL should migrate at about 25 kDa and the dimer human NGAL should migrate at about 50 kDa. The CHO cells expressing NGAL antigen demonstrated that about −80-90% of the expressed NGAL was monomer and ˜10-20% NGAL was dimer. The NGAL in the form of a dimer was converted to monomer under reducing conditions in β-mercaptoethanol containing loading buffer (See, FIG. 4).

### Iron Binding Assay

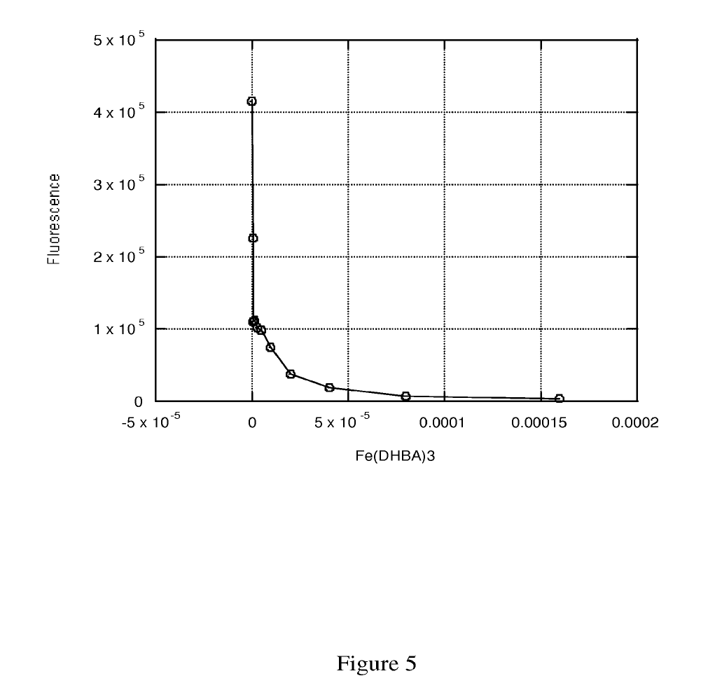

In order to further characterize the NGAL activity, its ability to bind Iron (III) dihydroxybenzoic (Fe(DHBA)₃) was measured. The binding of (Fe(DHBA)₃) results in the quenching of Trp fluorescence in NGAL. (Fe(DHBA)₃) is freshly prepared by incubating different amount of Fe³⁺ (0-160 μM) with an excess amount of DHBA (0.5 mM) in 0.1 M Tris, pH 7.5, at room temperature for 10 minutes. The mixtures are diluted 10-fold with TCN (0.5 mM Tris, 10 mM CaCl₂, 0.15 M NaCl, pH 7.5) and incubated with NGAL (50 μg/mL in TCN) in 2 mL centrifuge tubes at room temperature for 30 minutes (Goets, D. H. et al., Molecular Cell 10:1033 (2002)). The fluorescence is measured at 280 nm (excitation) and 340 nm (emission). The results demonstrated that human NGAL expressed from HEK293 produced as described above (transient expression) can bind >1.5 μM of (Fe(DHBA)₃) under the above conditions (See, FIG. 5).

### Western Blot Analysis

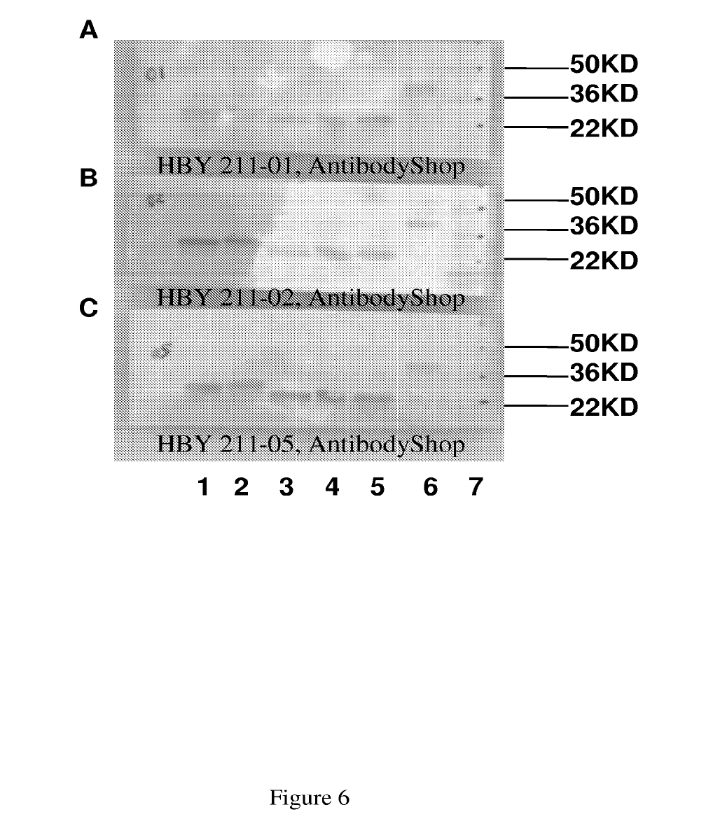

Approximately 3 μg of purified human recombinant NGAL protein was treated with SDS and 2-mercaptoethanol at 95° C. and electrophoresed in a 12% polyacrylamide-SDS gel (Laemmli et al., Nature, 227:680-685 (1970)). Proteins were transferred from the gel to nitrocellulose membranes by electrophoresis at 100 volts for 1-2 hours in a standard transfer buffer comprising 25 mM Tris ((Hydroxymethyl) Aminomethane), 192 mM glycine, and 2.0% methanol, pH 8.3 (Towbin et al., Proc. Natl. Acad. Sci., 73:4350-4354 (1979)). After transferring the proteins and blocking the nitrocellulose with 2% BSA in PBS, the nitrocellulose was used to determine the presence of human recombinant antigen. The nitrocellulose membrane was incubated with an appropriate amount of anti-NGAL monoclonal antibody (either HYB 211-01, HYB 211-02, or HYB 211-05, commercially available from AntibodyShop A/S, Gentofte, Denmark) in 10 ml of PBS/2% BSA buffer, pH 7.2. The nitrocellulose membranes were washed with phosphate buffered saline (PBS) pH 7.2, followed by addition of goat anti-mouse IgG antibody conjugated to HRP. The nitrocellulose membranes were incubated for one to two hours at room temperature, followed by washing with PBS. Finally, antibody bound to the protein was visualized by the addition of freshly prepared metal enhanced DAB in stable peroxide buffer (Pierce, Ill.). This assay demonstrated the anti-human NGAL monoclonal antibodies (AntibodyShop, Denmark) can bind to recombinant human NGAL antigen (See, FIG. 6).

### Glycosylation Analysis

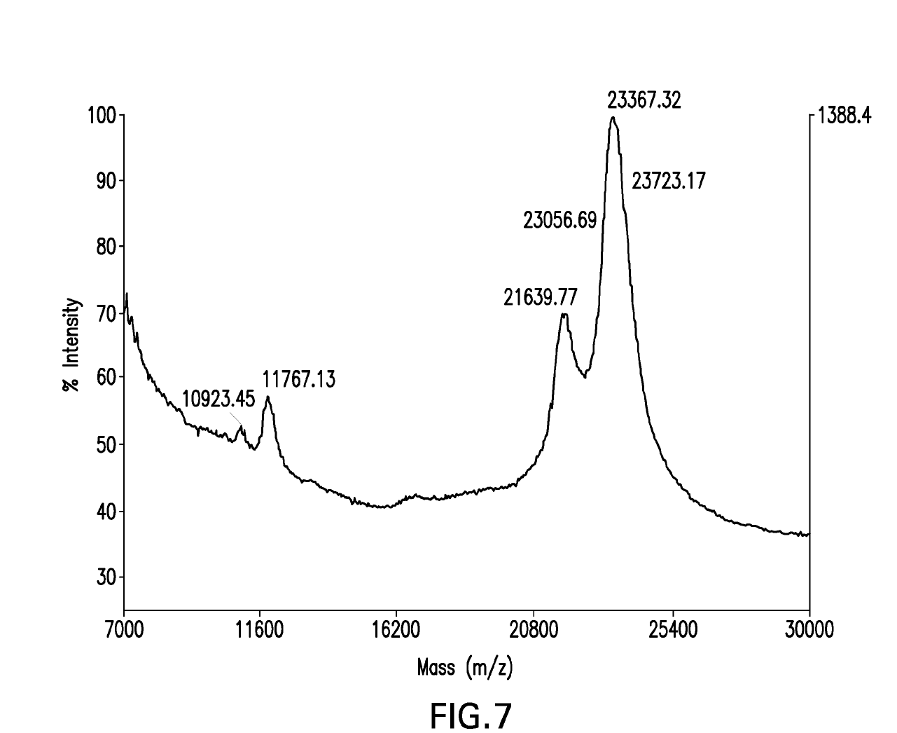

CHO cells expressing human NGAL antigen were analyzed by MALDI MS to determine N-linked glycosylation. The NGAL solution (20 μL) was dialyzed against 25 mM NH₄HCO₃, pH 8.0 using MiniDialysis tubes. To 10 μL solution, 0.5 PNGase (Sigma) was added and incubated at 37° C. for 20 or 72 hours. 0.2 μL of each of the above dialyzed samples was added to a sample spot on the MALDI sample plate. 0.2 μL of matrix solution (sinapinic acid solution in 1/1 (v/v) acetonitrile/water solution containing 0.25% TFA, prepared fresh) was mixed with the sample on the plate, air dried and loaded onto the MALDI instrument. An insulin solution (2 mg/mL in 0.1% TFA/H₂O) was loaded to a sample spot for instrument calibration. After 72 hours of treatment, part of the deglycosylated form of NGAL showed up as indicated by the appearance of a peak at 21639. This demonstrated there is N-linked glycan present on the CHO cells that expressed human NGAL (See, FIG. 7).

### Example 3

### Human NGAL Antigen Mutant C87S

### Human NGAL Mutant C87S Gene Cloning

Plasmid pJV-NGAL-A3 was used as template. Four PCR primers (two (2) sets of primer pairs, shown below) were designed to introduce serine replace cysteine at amino acid 87 of the wild-type human NGAL gene.

The first pair of primers used were as follows.

NGAL 5′-end primer (N-forsrf):
(SEQ ID NO: 5)
5′-CTT GCC CGG GCG CAC CAT GCC CCT AGG TCT CCT
G-3′.
NGAL 3′-end primer (NSer 2):
(SEQ ID NO: 7)
5′-GCT GCG AAC CTG GAA CAA AAG TCC TG-3′
(altered Serine codon underlined).

The second pair of primers used were as follows:

NGAL 5′-end primer (NSer 1):
(SEQ ID NO: 8)
5′-GTT CCA GGT TCG CAG CCC GGC GAG-3′
(altered Serine codon underlined).
NGAL 3′-end primer (N-revhis):
(SEQ ID NO: 6)
5′-CCC CGC GGC CGC TCA ATG GTG ATG GTG ATG ATG
GCC GTC GAT ACA CTG GTC GAT TGG-3′.

The first PCR reaction (NGAL-A) was done using the 5′-end primer (N-forsrf) of Example 1, which contained a partial NGAL signal sequence and a Srf I restriction site, and the 3′-end primer Nser2, which contained a serine codon instead of cysteine codon at amino acid 87. The PCR product from this primer pair was 341 bp. The second PCR reaction (NGAL-B) was done using the 5′-end primer (Nser1) set forth above, which contained a serine codon instead of cysteine codon at amino acid 87, and the 3′-end primer (N-revhis) of Example 1, which contained a Not I restriction site, 6×His and partial C-terminal NGAL sequence. The PCR product from this pair primer was 319 bp. The PCR reaction was executed in 2× reaction Buffer (dNTP), with the respective 5′ and 3′ primers (namely, one of the primer pairs above) and 1.25 units of Pfx DNA polymerase (Invitrogen Corp., Carlsbad, Calif.). The PCR was performed for 30 cycles of 15 seconds at 94° C. followed by 1 min at 68° C. A total of 30 cycles were performed. There is a 16-bp nucleotide overlap in primers Nser1 and Nser2.

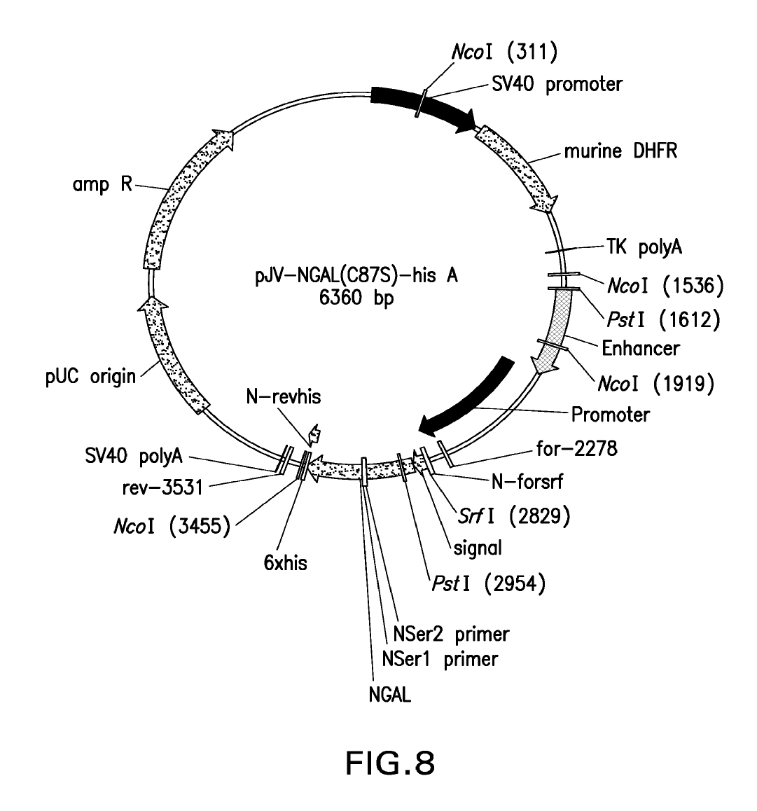

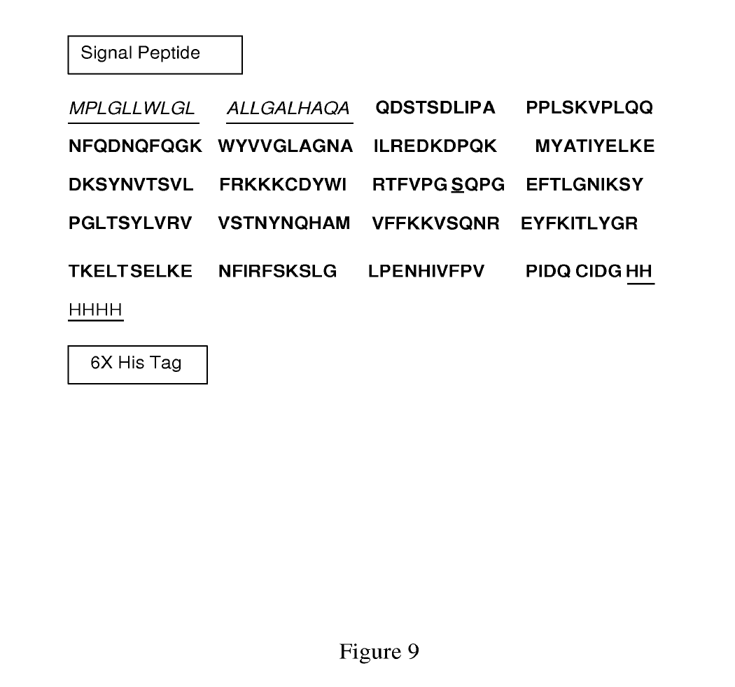

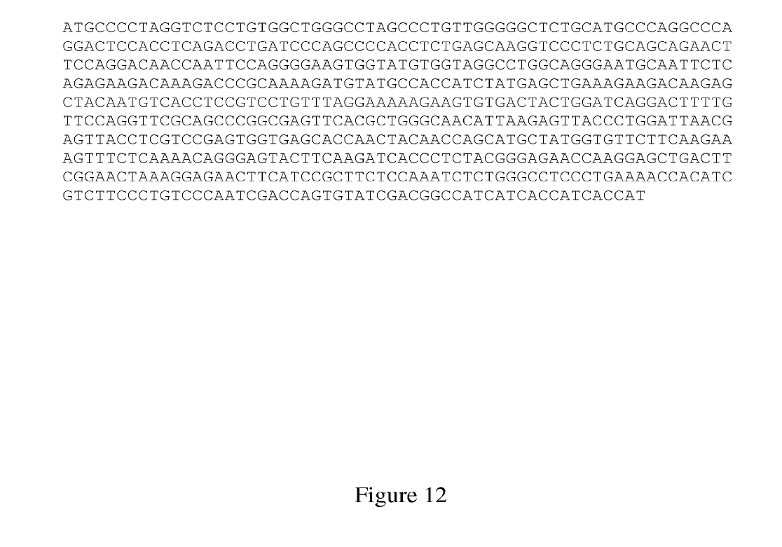

Two observed PCR products, namely NGAL-A (341 bp) and NGAL-B (319 bp) were gel purified and used as a template for another PCR reaction using the primer pair of N-forsrf and N-revhis (SEQ ID NOS:5 and 6 respectively). The PCR reaction was executed as described above. A full length human NGAL gene (643 bp) was amplified and restriction enzyme trimmed by Srf I and Not I, and then cloned into a pJV vector (as described in Example 1) and transformed into E. coli DH5α. The resulting pJV-based vector pJV-NGAL(ser87)-His-T3 (also known as pJV-NGAL(C87S)-his A; See in FIG. 8) comprises the ampicillin resistance gene, pUC origin, SV40 origin, EF-1a promoter, human NGAL signal peptide and full length human mutant NGAL DNA (SEQ ID NO:4 and FIG. 12 (human mutant NGAL DNA and FIG. 8)). The full length C87S mutant antigen sequence is shown in FIG. 9 (also, SEQ ID NO:2).

The transformed E. coli clones were grown in LB broth overnight with shaking at 37° C. Plasmid DNA was purified from each individual clone with the QIAprep spin miniprep kit (QIAGEN, Valencia, Calif.) followed by sequencing using the BigDye Terminator v3.1 cycle sequencing kit (Applied Biosystems, Foster City, Calif.). Plasmid pJV-NGAL(ser87)-His-T3 was selected by sequencing and analyzed by Vector NTI Advance™ software (Invitrogen Corp., Carlsbad, Calif.). Once the pJV clone was identified, separate E. coli DH5α cell banks containing pJV-NGAL(ser87)-His-T3 plasmid were made to preserve the pJV clones.

### Human NGAL C87S Mutant Antigen Transient Expression in HEK293 Cells

pJV-NGAL(ser87)-His-T3 plasmid DNA was Maxi prepared using Endofree plasmid Maxi kit (QIAGEN, Valencia, Calif.) by standard techniques. The high purity plasmid DNA obtained was then transiently transfected into HEK293 cells by 293 fectin (Invitrogen Corp., Carlsbad, Calif.). The transiently expressed human NGAL antigen C87S mutant was harvested and dia-filtrated as described in Example 1.

### Human NGAL C87S Mutant Antigen Purification

The dia-filtrated NGAL C87 mutant solution was purified using nickel-nitrilotriacetic acid (Ni-NTA, QIAGEN, Valencia, Calif.) metal-affinity chromatography as described in Example 1.

### Establishing a Stable NGAL C87S Mutant Expression CHO Cell Line and Methotrexate Amplification

A Chinese Hamster Ovary cell line (CHO, B3.2) that lacks the dihydrofolate reductase (DHFR) gene was used for transfection and stable human NGAL expression as in Example 1. The CHO cells were cultured and transfected by standard calcium phosphate-mediated transfection with the pJV-NGAL(ser87)-His-T3 plasmid. The transfected NGAL CHO cells were selected for several weeks in alpha MEM medium (Invitrogen Corp., Carlsbad, Calif.) lacking ribonucleosides and deoxyribonucleosides and containing 5% dialyzed FBS (dFBS) in a 10 cm tissue culture plate. After the transfected CHO cells had grown to more than 50% confluency, the transfected CHO cells went through a series of MTX amplifications in 20 nM, 250 nM, 500 nM, 2 μM and 5 μM MTX in a T flask. After a few weeks amplification, MTX amplified CHO cells were subcloned by end point dilution into alpha MEM+5% dFBS medium supplemented with 5 μM MTX in 96-well plates. The EIA assay was executed to rank the cell clones using anti-NGAL mouse monoclonal antibody coated on the EIA 96-well plates, followed by washing, and then incubating with supernatant from cultured CHO cell clones, then washing again and incubating with biotin labeled goat-anti NGAL (R&D Systems, Minneapolis, Minn.), then washing again and incubating with streptavidin (SA)-HRP for another 30 minutes to 1 hour and then finally developing by OPD as described in Example 1. A human NGAL rAg C87S mutant CHO cell clone #734 was identified and weaned into serum-free medium, i.e., DHFR-CHO medium (Sigma-Aldrich, St. Louis, Mo.). A cell bank of NGAL rAg CHO cell clone #734 was established.

### Example 4

### Characterization of Recombinant C87S Mutant NGAL Antigen

### SDS-PAGE Gel Electrophoresis

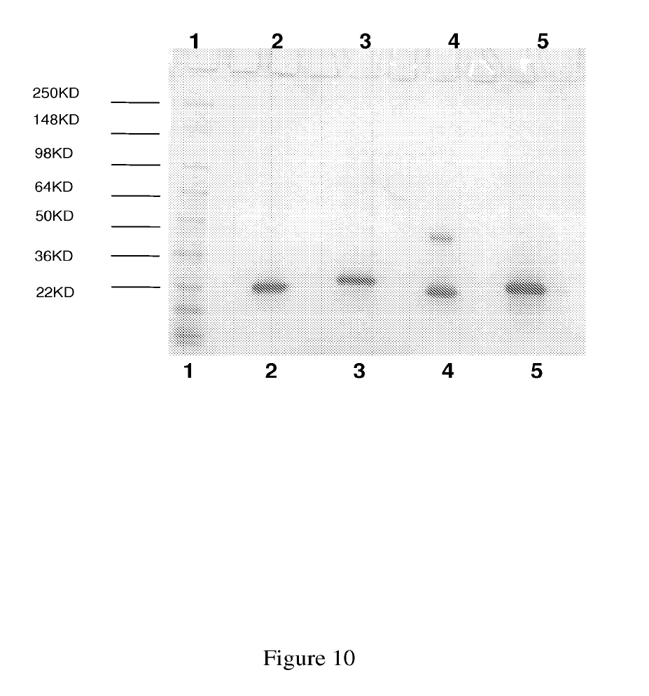

SDS-PAGE gel electrophoresis was performed on CHO expressed human C87S mutant NGAL recombinant antigen under reducing conditions or non-reducing conditions. About 3 μg of C87S mutant NGAL antigen was mixed with loading buffer with or without reducing agents (β-mercaptoethanol), boiled for 10 minutes, then loaded onto a 4-20% SDS-PAGE gel and run at 80 Volts for 1.5 hours. The monomer human NGAL should migrate at about 25 kDa, and the dimer human NGAL should migrate at about 50 kDa. The CHO cells expressing C87S mutant NGAL antigen demonstrated that about >95% NGAL is in monomer form (with or without reducing agent added). No dimer human NGAL was found to be present in this mutant preparation (See, FIG. 10).

### Example 5

### ATCC Deposit Information

The wild-type NGAL rAg CHO 662 cell line was deposited with the American Type Culture Collection (ATCC) at 10801 University Boulevard, Manassas, Va. 20110-2209 on Nov. 21, 2006 and received ATCC Accession No. PTA-8020.

The mutant NGAL rAg CHO C87S cell line (CHO cell clone #734, also known as “mutant C87S NGAL rAg CHO 734”) was deposited with the American Type Culture Collection (ATCC) at 10801 University Boulevard, Manassas, Va. 20110-2209 on Jan. 23, 2007 and received ATCC Accession No. PTA-8168.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference. In particular, the following two U.S. Patent applications, each filed Oct. 19, 2007, are incorporated by reference in their entireties: U.S. Provisional Application Ser. Nos. 60/981,471 and 60/981,473.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as encompassed by the appended claims. Moreover, it should be understood that where certain terms are defined under “Definitions” and are otherwise defined, described, or discussed elsewhere in the “Detailed Description,” all such definitions, descriptions, and discussions are intended to be attributed to such terms. There also is no intention in the use of such terms and expressions of excluding any equivalents of the features shown and described or portions thereof. Furthermore, while subheadings, e.g., “Definitions,” are used in the “Detailed Description,” such use is solely for ease of reference and is not intended to limit any disclosure made in one section to that section only; rather, any disclosure made under one subheading is intended to constitute a disclosure under each and every other subheading.

What is claimed is:

1. A cell line selected from the group consisting of a Chinese Hamster Ovary (CHO) cell line which produces glycosylated mammalian NGAL and a human embryonic kidney (HEK) cell line which produces glycosylated mammalian NGAL.

2. The CHO cell line or HEK cell line of claim 1, wherein the mammalian glycosylated NGAL is selected from the group consisting of: canine, feline, rat, murine, horse, non-human primates and humans.

3. The CHO cell line or HEK cell line of claim 1, wherein said glycosylated mammalian NGAL is wild-type human NGAL.

4. The CHO cell line or HEK cell line of claim 3, wherein said wild-type human NGAL comprises the amino acid sequence of SEQ ID NOS:1 or 12.

5. The CHO cell line or HEK cell line of claim 3, wherein said CHO cell line is ATCC Accession No. PTA-8020.

6. The CHO cell line or HEK cell line of claim 3, wherein said glycosylated wild-type human NGAL comprises a molecular weight of about 25 kDa.

7. The CHO cell line or HEK cell line of claim 1, wherein said glycosylated mammalian NGAL comprises an amino acid sequence that comprises one or more amino acid substitutions, deletions, or additions when compared to the amino acid sequence of wild-type mammalian NGAL.

8. The CHO cell line or HEK cell line of claim 1, wherein said glycosylated mammalian NGAL is human NGAL, and further said human NGAL comprises an amino acid substitution at the amino acid corresponding to amino acid 87 of the amino acid sequence of wild-type human NGAL set forth in SEQ ID NOS:1 or 12.

9. The CHO cell line or HEK cell line of claim 8, wherein said amino acid substitution comprises replacement of a cysteine with a serine.

10. The CHO cell line or HEK cell line of claim 9, wherein said glycosylated human NGAL comprises the amino acid sequence of SEQ ID NOS:2 or 10.

11. The CHO cell line of claim 9, wherein said CHO cell line is ATCC Accession No. PTA-8168.

12. A method of producing glycosylated mammalian NGAL, said method comprising the steps of:
(a) transfecting a cell line with a gene encoding mammalian NGAL under conditions such that glycosylated mammalian NGAL is produced; and
(b) recovering said glycosylated mammalian NGAL produced by said cell line.

13. The method of claim 12, wherein the glycosylated mammalian NGAL is selected from the group consisting of: canine, feline, rat, murine, horse, non-human primates and humans.

14. The method of claim 12, wherein the glycosylated mammalian NGAL is human NGAL.

15. The method of claim 12, wherein said cell line comprises Chinese Hamster Ovary (CHO) cells.

16. The method of claim 12, further comprising in step (a) transfecting said cell line with an amplification gene, carrying out selection for amplified cells, and then carrying out step (b).

17. The method of claim 16, wherein the amplification gene encodes dihydrofolate reductase or glutamine synthase, and selection is done with methotrexate or glutamine.

18. The method of claim 12, wherein said glycosylated human NGAL comprises wild-type human NGAL.

19. The method of claim 18, wherein said wild-type human NGAL comprises the amino acid sequence of SEQ ID NOS:1 or 12.

20. The method of claim 18, wherein said glycosylated human NGAL comprises a molecular weight of about 25 kDa.

21. The method of claim 12, wherein said glycosylated human NGAL comprises an amino acid sequence that comprises one or more amino acid substitutions, deletions or additions when compared to the amino acid sequence of wild-type human NGAL.

22. The method of claim 12, wherein said glycosylated human NGAL comprises an amino acid substitution at the amino acid corresponding to amino acid 87 of the amino acid sequence of wild-type human NGAL.

23. The method of claim 22, wherein said amino acid substitution comprises replacement of a cysteine with a serine.

24. The method of claim 23, wherein said glycosylated human NGAL comprises the amino acid sequence of SEQ ID NOS:2 or 10.

25. Glycosylated human NGAL produced by the method of claim 12, wherein said human NGAL comprises a sequence selected from the group consisting of SEQ ID NO:1, SEQ ID NO: 12., SEQ ID NO:2, and SEQ ID NO:10.

26. An isolated mutant glycosylated human NGAL comprising the sequence of SEQ ID NOS:2 or 10.

27. A calibrator or control for use in an assay for detecting mammalian NGAL in a test sample, said calibrator or control comprising glycosylated mammalian NGAL.

28. The calibrator or control of claim 27, wherein the mammalian NGAL is canine, feline, rat, murine, horse, non-human primates and humans.

29. The calibrator or control of claim 27, wherein the mammalian NGAL is glycosylated human NGAL comprising the sequence selected from the group consisting of SEQ ID NOS:2, SEQ ID NO:10, SEQ ID NO:1 and SEQ ID NO:12.

30. The calibrator or control of claim 27, wherein the method is adapted for use in an automated system or semi-automated system.

31. A method of preventing or eliminating the formation of at least one dimer of human NGAL in a calibrator, control or other sample, said method comprising introducing an amino acid substitution into said human NGAL which comprises replacement of cysteine with serine at the amino acid corresponding to amino acid 87 of the amino acid sequence of wild-type human NGAL set forth in SEQ ID NOS:1 or 12.

32. The method of claim 31, wherein the dimer is a homodimer.

33. The method of claim 31, wherein the dimer is heterodimer.

34. An isolated and purified human NGAL polynucleotide comprising the sequence of SEQ ID NOS:4 or 11.


SEQUENCE LISTING (12 sequences)

SEQ ID NO: 1 - protein, 198 aa, Homo sapiens
  MISC_FEATURE (1)..(20): The first 20 amino acids are the signal peptide which would be labeled -1 to -20, with Gln (at position 21) being considered amino acid 1 of the NGAL peptide
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 198 aa, Artificial sequence
  Chemically synthesized
MPLGLLWLGLALLGALHAQAQDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - DNA, 612 bp, Homo sapiens
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttgccagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggccatcatcaccatcaccat

SEQ ID NO: 4 - DNA, 612 bp, Artificial sequence
  Chemically synthesized
atgcccctaggtctcctgtggctgggcctagccctgttgggggctctgcatgcccaggcccaggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttcgcagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggccatcatcaccatcaccat

SEQ ID NO: 5 - DNA, 34 bp, Artificial sequence
  Chemically synthesized
cttgcccgggcgcaccatgcccctaggtctcctg

SEQ ID NO: 6 - DNA, 57 bp, Artificial sequence
  Chemically synthesized
ccccgcggccgctcaatggtgatggtgatgatggccgtcgatacactggtcgattgg

SEQ ID NO: 7 - DNA, 26 bp, Artificial sequence
  Chemically synthesized
gctgcgaacctggaacaaaagtcctg

SEQ ID NO: 8 - DNA, 24 bp, Artificial sequence
  Chemically synthesized
gttccaggttcgcagcccggcgag

SEQ ID NO: 9 - DNA, 558 bp, Artificial sequence
  Chemically synthesized
atgcaggactctacttccgacctgattccggctccgccgctgtctaaagtgccgctgcagcagaactttcaagacaaccagttccagggtaaatggtacgttgtgggcctggctggtaacgcgatcctgcgtgaagacaaagatccgcagaaaatgtatgctaccatctacgaactgaaagaagacaaatcttataacgtgaccagcgttctgtttcgtaaaaagaaatgtgactactggattcgcaccttcgtgccgggctctcagccgggcgagttcactctgggtaacatcaaatcttacccgggtctgactagctacctggtgcgtgtggtttctactaactataaccagcatgctatggtgttcttcaagaaagtttctcagaaccgtgaatacttcaagattactctgtacggtcgtaccaaagagctgacctctgagctgaaagaaaacttcatccgtttctctaaatctctgggcctgccggagaaccatatcgtgtttccggttccgatcgatcagtgcatcgacggtcatcatcaccatcaccattga

SEQ ID NO: 10 - protein, 178 aa, Artificial sequence
  Chemically synthesized
QDSTSDLIPAPPLSKVPLQQNFQDNQFQGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 11 - DNA, 552 bp, Artificial sequence
  Chemically synthesized
caggactccacctcagacctgatcccagccccacctctgagcaaggtccctctgcagcagaacttccaggacaaccaattccaggggaagtggtatgtggtaggcctggcagggaatgcaattctcagagaagacaaagacccgcaaaagatgtatgccaccatctatgagctgaaagaagacaagagctacaatgtcacctccgtcctgtttaggaaaaagaagtgtgactactggatcaggacttttgttccaggttcgcagcccggcgagttcacgctgggcaacattaagagttaccctggattaacgagttacctcgtccgagtggtgagcaccaactacaaccagcatgctatggtgttcttcaagaaagtttctcaaaacagggagtacttcaagatcaccctctacgggagaaccaaggagctgacttcggaactaaaggagaacttcatccgcttctccaaatctctgggcctccctgaaaaccacatcgtcttccctgtcccaatcgaccagtgtatcgacggccatcatcaccatcaccat

SEQ ID NO: 12 - protein, 178 aa, Artificial sequence
  Chemically synthesized
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG